(19) United States
(12) Patent Application Publication
(10) Pub. No.: US 2007/0255333 A1
(43) Pub. Date: Nov. 1, 2007

(54) Neuromodulation therapy for perineal or dorsal branch of pudendal nerve

(75) Inventors: Jonathon E. Giftakis, Maple Grove, MN (US); Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/414,509

(22) Filed: Apr. 28, 2006

(51) Int. Cl. A61N 1/18

(52) U.S. Cl. 607/39; 607/46; 607/40

(57) ABSTRACT

The disclosure describes a method and system for applying electrical stimulation to a pudendal nerve of a patient via an implantable electrical stimulation device to treat a pelvic disorder in men or women. Pelvic disorders may include sexual dysfunction, urinary incontinence, pudendal nerve entrapment (PNE), and urogenital pain or other forms of pelvic pain, e.g., chronic pelvic pain and prostatitis-like pain. The electrical stimulation may be applied to one or both pudendal nerves and, more particularly, to at least one of a dorsal branch and a perineal branch of one or both pudendal nerves. In some embodiments, the electrical stimulation may be applied to at least one of the dorsal and perineal branches of a pudendal nerve via a pudendal canal of the patient. In further embodiments, drug therapy may be delivered alone or in combination with electrical stimulation to one or both pudendal nerves of a patient via an implantable drug delivery device.

### TECHNICAL FIELD

The invention relates to implantable medical devices and, more particularly, to devices for delivering neuromodulation therapy to treat pelvic floor disorders.

### BACKGROUND

Pelvic floor disorders adversely affect the health and quality of life of millions of people. Pelvic floor disorders include urinary control disorders, sexual dysfunction, and pelvic pain. Pelvic floor disorders can be treated with a variety of therapeutic options such as behavior modification including biofeedback, pharmacological treatment, mechanical intervention such as self-catheterization, physical appliances such as diapers, and surgical intervention. Surgical treatments are the most invasive and are often considered after other therapies have proven ineffective.

Urinary incontinence, or an inability to control urinary function, is a common problem afflicting people of all ages, genders, and races. Individuals with urinary control disorders often face debilitating challenges in their everyday lives. These individuals can be preoccupied with trips to the bathroom, fears of embarrassment, and sleepless nights. Some sufferers become so anxious that they become isolated and depressed. Various muscles, nerves, organs and conduits within the urinary tract cooperate to collect, store and release urine. A variety of disorders may compromise urinary tract performance and contribute to incontinence. Although there are a variety of different types of urinary incontinence, stress incontinence, urge incontinence and urinary retention are the most common. Many of the disorders may be associated with aging, injury or illness.

Sexual dysfunctions plague both women and men, and may be life-long or acquired. Sexual dysfunction comprises a broad range of maladies, including erectile dysfunction, orgasmic dysfunction, premature ejaculation and lack of lubrication. In women, sexual dysfunction includes desire, arousal, orgasmic and sex pain disorders (dyspareunia and vaginismus). In men, sexual dysfunction of the penis is a common problem afflicting males of all ages, genders, and races. Erectile dysfunction is a serious condition for many men, and it may include a variety of problems. Some of these problems include the inability to create an erection, incomplete erections and brief erectile periods. These conditions may be associated with nervous system disorders and may be caused by aging, injury, or illness.

In some cases, erectile dysfunction can be attributed to improper nerve activity that incompletely stimulates the penis. For example, stimulation from the brain during arousal and sexual activity is responsible for activating an erection. With respect to erectile disorders, the problem may be a lack of sufficient stimulation from the brain or a break in communication of the stimulation. Other disorders may involve dysfunctional parasympathetic function that can be attributed to many factors including illness or injury.

Some methods for treating erectile dysfunction include pharmaceutical treatment and electrical stimulation. Delivery of electrical stimulation to nerves running through the pelvic floor may provide an effective therapy for many patients. For example, an implantable neurostimulator may be provided to deliver electrical stimulation to the pudendal or cavernous nerve to induce an erection.

Pain in the pelvic region, including urogenital pain, may be caused by a variety of injuries or disorders in men and women. For example, pudendal nerve entrapment (PNE), chronic groin pain, chronic testicular pain (CTP), urogenital pain, prostatitis-like pain, and other pain originating from the pelvic or groin region are common reasons for referral to a urological specialist. Typically, pain is worsened by sitting, and can include prickling, stabbing, burning, numbness, and a sense of a foreign object in the urethra, vagina (in women), or rectum. In addition to pain, symptoms of PNE can include sexual dysfunction.

As an example, pudendal nerve entrapment (PNE), chronic groin pain, chronic testicular pain (CTP), urogenital pain, and prostatitis-like pain, may be attributed to nerve injury, such as compression of a nerve by impact trauma, iatrogenic injury, entrapment of the nerve in scar tissue, irritation because of proximity to a zone of inflammation, childbirth, bicycling or other activities that require rigorous climbing and/or squatting (football, wresting, weightlifting, and the like), or congenital deformations. Iatrogenic injury may be caused by various surgical procedures such as radical perineal prostatectomy.

Various methods may be used to treat PNE, chronic groin pain, chronic testicular pain (CTP), urogenital pain, prostatitis-like pain, and other pain originating from the pelvic or groin region. As an example, pharmaceutical treatment, e.g., antibiotics, anti-inflammatory agents, alpha blockers, anti-spasmodics, analgesics, allopurinol, and muscle relaxants, may be effective, but the patient may require progressively increased dosages as his body adapts to the treatment. Denervation procedures may also be used to treat PNE, chronic groin pain, chronic testicular pain (CTP), urogenital pain, and prostatitis-like pain. In denervation procedures, the nerve that is diagnosed, e.g., using the results of the patient history, physical examination, preoperative electromyography, and nerve blocks, as the cause is severed or permanently removed. Such procedures may result in permanent and substantial pain relief. However, severing or removing some nerves may result in sexual dysfunction, urinary incontinence, and loss of sensation. Therapeutic nerve blocks may also be used to treat PNE, chronic groin pain, chronic testicular pain (CTP), urogenital pain, and prostatitis-like pain, but generally only relieve pain temporarily.

### SUMMARY

In general, the invention is directed to techniques for applying neuromodulation therapy to a perineal branch and/or dorsal branch of a pudendal nerve of a patient via an implantable medical device to treat a pelvic disorder in men or women. Neuromodulation therapy refers to electrical stimulation, drug (or other fluid agent) delivery, or a combination of both, to one or more nerve sites to block, attenuate, generate, or amplify nerve signals. Pelvic disorders may include sexual dysfunction, urinary incontinence, pudendal nerve entrapment (PNE), and urogenital pain or other forms of pelvic pain, e.g., chronic pelvic pain and prostatitis-like pain. Neuromodulation therapy in the form of electrical stimulation and/or drug delivery may be applied to perineal and/or dorsal branches of one or both pudendal nerves, e.g., on a unilateral (one pudendal nerve) or bilateral basis (both pudendal nerves). In some embodiments, the neuromodulation therapy may be applied to at least one of the dorsal and perineal branches of a pudendal nerve either directly or via a pudendal canal of the patient.

A system according to the invention may include one or more electrical stimulators that apply electrical stimulation to at least one of a dorsal branch and a perineal branch of the pudendal nerve to treat one or more pelvic disorders, such as sexual dysfunction, urinary incontinence, PNE, pelvic pain, or other afflictions associated with pain originating from the pelvic or groin regions. The electrical stimulators may comprise various types of electrodes such as ring electrodes, cuff electrodes, paddle lead electrodes and/or microstimulators implanted at various locations proximate to one or both of the pudendal nerves of a patient.

The electrical stimulators may be implanted proximate to at least one of the dorsal and perineal branches at a point prior to entering a pudendal canal of a patient or at a point after the dorsal or perineal branch exits the pudendal canal. Additionally or alternatively, electrical stimulators may be implanted proximate to the pudendal canal to deliver electrical stimulation to at least one of the dorsal and perineal branches of the pudendal nerve via the pudendal canal. Stimulation may be applied uni-laterally, i.e., via at least one branch of the pudendal nerve, or bi-laterally, i.e., via at least one branch of both pudendal nerves.

In some embodiments, electrical stimulation electrodes may be coupled to an implantable stimulation device implanted within a subcutaneous pocket in the abdomen of the patient or, alternatively, the scrotum or buttock of the patient. The electrical stimulation electrodes may be coupled to the implantable medical device via standard implantable electrode leads. Alternatively, leadless microstimulators may be positioned adjacent the target nerves. In this case, the leadless microstimulators may be capable of wireless communication with other implantable medical devices, an external programmer, or both.

Stimulation electrodes or leadless microstimulators may be implanted using well known surgical procedures such as those used in exposing the pudendal nerve, implanting stimulation electrodes for treating sexual dysfunction, or pudendal denervation. Systems including such electrodes or microstimulators and employing the techniques described in this disclosure may substantially reduce or eliminate chronic pelvic pain, including urogenital pain such as chronic groin pain, chronic testicular pain (CTP), urogenital pain, prostatitis-like pain, or pain associated with PNE without loss of sensation in the penis or scrotum or other unwanted side effects, such as sexual dysfunction and urinary incontinence.

In some embodiments, drug therapy may be applied by an implantable medical device alone or in combination with electrical stimulation. Accordingly, a system according to the invention may include, in addition to an electrical stimulation device, one or more fluid transfer devices, such as a catheter, a conduit, or the like, to transfer the drug from a reservoir to the delivery site, and a pump coupling the reservoir to the fluid transfer devices that pumps the drug from the reservoir to the delivery site via the fluid transfer devices. The implantable drug delivery device may be incorporated with the electrical stimulation device in a single device, i.e., in a common implantable medical device, or may be independent of the electrical stimulation device.

In some embodiments, the drug delivery device may be capable of delivering one or more drugs and, accordingly, may include more than one reservoir. Each reservoir may contain a drug or a mixture of drugs. The drug delivery device may also include a processor that controls the function of the drug delivery device to, for example, control which of a plurality of drugs contained in the drug delivery device are delivered and the dosage of the drugs delivered. The fluid transfer devices may be implanted in a similar fashion as the electrical stimulators, i.e., at various locations proximate to at least one of a dorsal and perineal branches of one or both perineal nerves of a patient. The drug may be selected to treat sexual dysfunction or pelvic pain, such as chronic groin pain, chronic testicular pain (CTP), urogenital pain, prostatitis-like pain, or pain associated with PNE.

Systems according to the invention may include an external programmer that programs the electrical stimulators to apply electrical stimulation to a dorsal or perineal branch of the pudendal nerve. During stimulation, a clinician or patient may operate the external programmer to adjust stimulation parameters, such as amplitude, pulse width, pulse rate, and electrode polarities. In some cases, a patient may use the programmer to deliver stimulation on demand, e.g., when the patient experiences discomfort. Additionally or alternatively, the implantable stimulation device may store stimulation programs and schedules. In this manner, the electrical stimulation can be delivered according to preprogrammed stimulation parameters and schedules, if desired.

In embodiments in which the system delivers drug therapy in combination with electrical stimulation, a clinician or patient may similarly operate the external programmer to adjust drug delivery parameters, such as which of a dosage or rate of delivery of a drug, or which of a plurality of drugs contained in the device are delivered, and/or deliver drug therapy on demand. In such embodiments, the implantable stimulation device may store drug therapy programs and schedules and deliver drug therapy according to preprogrammed stimulation parameters and schedules.

In one embodiment, the invention provides a method comprising applying electrical stimulation to at least one branch of a pudendal nerve of a patient via an implanted electrical stimulation device.

In another embodiment, the invention provides a system comprising an implantable electrical stimulation device that generates electrical stimulation selected to treat a pelvic disorder, and one or more electrodes coupled to the electrical stimulation device at a position adjacent to at least one of a dorsal branch and a perineal branch of a pudendal nerve of a patient.

In an additional embodiment, the invention provides a method comprising delivering electrical stimulation to at least one of a dorsal branch and a perineal branch of at least one pudendal nerve of a patient via an implanted electrical stimulation device, and delivering a fluid to at least at least one of the dorsal and perineal branches of the pudendal nerves of the patient via an implanted fluid delivery device, wherein the implanted fluid delivery device and the implanted fluid delivery device share a common housing.

In a further embodiment, the invention provides a system comprising an implantable electrical stimulation device that delivers electrical stimulation selected to alleviate a pelvic disorder to at least one of a dorsal branch and a perineal branch of at least one pudendal nerve of a patient, and an implantable fluid delivery device that delivers a fluid selected to alleviate a pelvic disorder to at least one of the dorsal and perineal branches of at least one pudendal nerve of the patient, wherein the implanted electrical stimulation device and the implanted fluid delivery device share a common housing.

In another embodiment, the invention provides a method comprising delivering a fluid to at least at least one of the dorsal and perineal branches of the pudendal nerves of the patient via an implanted fluid delivery device.

In an additional embodiment, the invention provides a system comprising an implantable fluid delivery device that containing a fluid selected to alleviate a pelvic disorder, and a catheter, coupled to the implantable fluid delivery device, that delivers the fluid to at least one of the dorsal and perineal branches of at least one pudendal nerve of the patient.

In various embodiments, the invention may provide one or more advantages. For example, applying electrical stimulation to at least one of a dorsal branch and a perineal branch of a pudendal nerve of a patient may substantially reduce or eliminate sexual dysfunction, urinary incontinence, and pelvic pain such as that associated with PNE, chronic groin pain, chronic testicular pain (CTP), urogenital pain, and prostatitis-like pain.

Denervation procedures that sever or remove a portion of the pudendal nerve often result in unwanted side effects including loss of sensation in the skin of the scrotum and the penis, sexual dysfunction, and urinary incontinence. Therapeutic nerve blocks typically only relieve pain temporarily. In contrast, delivery of a electrical stimulation and/or drug therapy to at least one of the dorsal and perineal branches of one or both pudendal nerves may provide permanent or long-lived effective therapy for many patients with fewer or no unwanted side effects.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating an example system that includes an implantable stimulation device for applying electrical stimulation to at least one of a dorsal branch and a perineal branch of a pudendal nerve of a patient to treat a pelvic disorder from a front view of a male patient.

FIG. 2 is a schematic diagram further illustrating the example system of FIG. 1 from a top view of a male patient.

FIG. 3 is a schematic diagram illustrating another exemplary configuration of the system of FIG. 1 from a side view of a male patient.

FIG. 4 is a schematic diagram illustrating another exemplary configuration of the system of FIG. 1 from a side view of a male patient.

FIG. 5 is a schematic diagram illustrating a further configuration of the system of FIG. 1 from a side view of male patient.

FIG. 6 is a block diagram illustrating an example implantable stimulation device for applying electrical stimulation to a branch of a pudendal nerve of a patient.

FIG. 7 is a block diagram illustrating an example clinician programmer that allows a clinician to program electrical stimulation therapy for a patient.

FIG. 8 is a schematic diagram illustrating an example system that includes an implantable medical device for delivering drug therapy in combination with electrical stimulation to at least one of a dorsal branch and a perineal branch of a pudendal nerve of a patient to treat a pelvic disorder from a front view of a male patient.

FIG. 9 is a schematic diagram illustrating another configuration for the example system of FIG. 8.

FIGS. 10A and 10B are schematic diagrams illustrating incorporation of fixation elements in an electrode lead or fluid transfer device.

FIG. 11 is a schematic diagram further illustrating the example system of FIG. 8 with a different type of electrical stimulator from a side view of a male patient.

FIGS. 12A-12C are schematic diagrams illustrating an example cuff electrode useful in the system of FIG. 1.

FIG. 13 is a schematic diagram further illustrating the example system of FIG. 8 with another type of electrical stimulator from a side view of a male patient.

FIGS. 14A-14C are schematic diagrams illustrating an example leadless microstimulator suitable for use in the system of FIG. 13.

FIG. 15 is a side cross-sectional view of a leadless electrical microstimulator implanted within tissue proximate to a branch of a pudendal nerve of a patient.

FIG. 16 is a schematic diagram illustrating implantation of a leadless microstimulator within an Pudendal canal of a patient or within tissue proximate to a dorsal or perineal branch of a pudendal nerve of a patient.

FIG. 17 is a functional block diagram illustrating various components of the leadless microstimulator of FIG. 15.

FIG. 18 is a flow chart illustrating a technique for applying electrical stimulation to at least one of a dorsal branch and a perineal branch of a pudendal nerve of a patient to treat a pelvic disorder.

### DETAILED DESCRIPTION

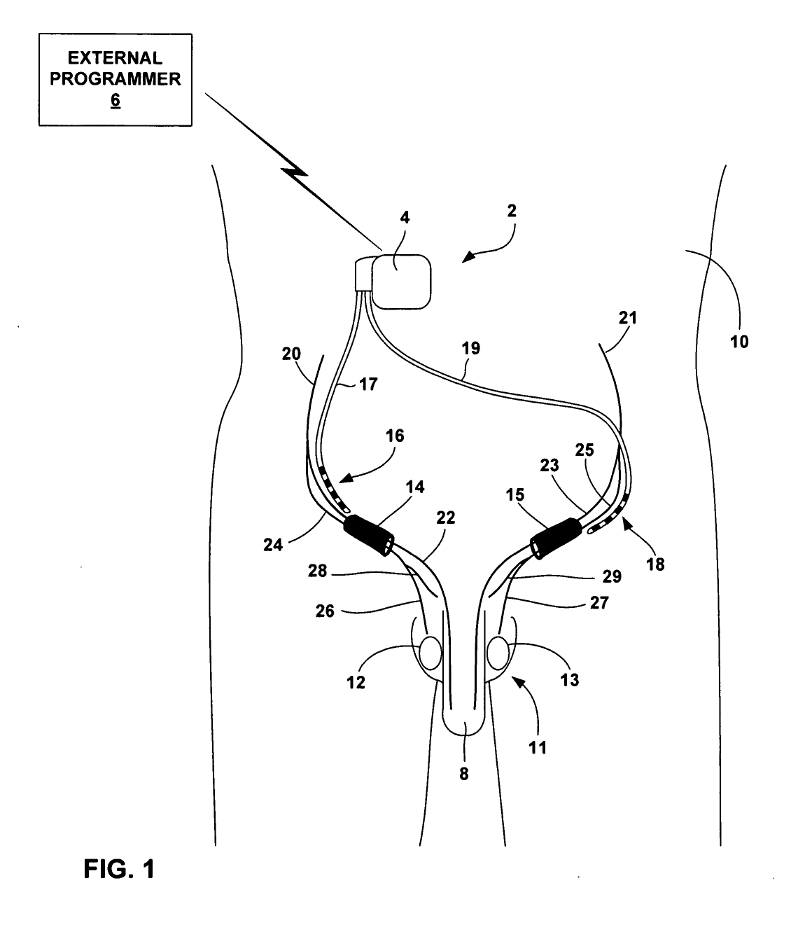

FIG. 1 is a schematic diagram illustrating an example system 2 that includes an implantable medical device (IMD) 4 in the form of an electrical stimulator that applies electrical stimulation to at least one branch of one or both pudendal nerves of a patient 10. In FIG. 1, system 2 is illustrated from a front view perspective of patient 10. Although the invention may be generally applicable to treat pelvic pain in both men and women, application of the invention to men will be described throughout this disclosure for purposes of illustration. Throughout the figures accompanying this disclosure, various anatomical features of patient 10 and structural features of system 2 are illustrated conceptually for ease of illustration. Accordingly, the figures may not necessarily present appropriate scales and proportions of such anatomical features. Rather, the drawings are provided as a conceptual rendering of such features to aid in the understanding of pertinent embodiments of the invention.

In the example of FIG. 1, IMD 4 applies electrical stimulation to patient 10 to treat one or more pelvic disorders, such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region. IMD 4 may also apply electrical stimulation to patient 10 for alleviation of chronic pelvic pain that is idiopathic in origin.

Sexual dysfunctions plague both women and men, and may be life-long or acquired. Sexual dysfunction comprises a broad range of maladies, including erectile dysfunction, orgasmic dysfunction, premature ejaculation and lack of lubrication. In women, sexual dysfunction includes desire, arousal, orgasmic and sex pain disorders (dyspareunia and vaginismus). In men, sexual dysfunction of the penis is a common problem afflicting males of all ages, genders, and races. Erectile dysfunction is a serious condition for many men, and it may include a variety of problems. Some of these problems include the inability to create an erection, incomplete erections and brief erectile periods. These conditions may be associated with nervous system disorders and may be caused by aging, injury, or illness.

In some cases, erectile dysfunction can be attributed to improper nerve activity that incompletely stimulates the penis. For example, stimulation from the brain during arousal and sexual activity is responsible for activating an erection. With respect to erectile disorders, the problem may be a lack of sufficient stimulation from the brain or a break in communication of the stimulation. Other disorders may involve dysfunctional parasympathetic function that can be attributed to many factors including illness or injury.

Urinary incontinence, or an inability to control urinary function, is a common problem afflicting people of all ages, genders, and races. Individuals with urinary control disorders often face debilitating challenges in their everyday lives. These individuals can be preoccupied with trips to the bathroom, fears of embarrassment, and sleepless nights. Some patients become so anxious they become isolated and depressed. Various muscles, nerves, organs and conduits within the urinary tract cooperate to collect, store and release urine. A variety of disorders may compromise urinary tract performance and contribute to incontinence. Although there are a variety of different types of urinary incontinence, stress incontinence, urge incontinence and urinary retention are the most common. Many of the disorders may be associated with aging, injury or illness.

Pain in the pelvic region, including urogenital pain, chronic groin pain, chronic testicular pain (CTP), prostatitis-like pain, and pain associated with PNE or other forms of pelvic pain that cause chronic pain in the pelvic or groin region may be caused by a variety of injuries or disorders in men and women. Typically, pain is worsened by sitting, and can include prickling, stabbing, burning, numbness, and a sense of a foreign object in the urethra, vagina (in women), or rectum. In addition to pain, symptoms of PNE can include sexual dysfunction. As an example, PNE, chronic groin pain, chronic testicular pain (CTP), and prostatitis-like pain, may be attributed to nerve injury, such as compression of a nerve by impact trauma, iatrogenic injury, entrapment of the nerve in scar tissue, irritation because of proximity to a zone of inflammation, childbirth, bicycling, or other activities that require rigorous climbing and/or squatting, e.g., football, wrestling, weightlifting, and the like), or congenital deformations. Iatrogenic injury may be caused by various surgical procedures such as radical perineal prostatectomy.

FIG. 1 illustrates pudendal nerves 20, 21 and dorsal branches 22, 23 and perineal branches 24, 25 of pudendal nerves 20, 21, respectively. Pudendal canals 14, 15 include a portion of dorsal nerve branches 22, 23 and perineal nerve branches 24, 25 of pudendal nerves 20, 21, respectively. Generally, IMD 4 delivers electrical stimulation to at least one of dorsal nerve branches 22, 23 and perineal nerve branches 24, 25 via electrodes which may be coupled to IMD 4 by one or more leads. The electrical stimulation has parameters selected to block or attenuate pain signals from the pelvic and/or groin region, including penis 8 and scrotal skin 11 from reaching the central nervous system (CNS). The electrodes may be implanted at various locations proximate to dorsal branches 22, 23 or perineal branches 24, of pudendal nerves 20, 21, respectively, including at positions above, below or adjacent to pudendal canals 14, 15.

As shown in the illustrated example of FIG. 1, electrodes may be implanted proximate to a portion of dorsal nerve branches 22, 23 or perineal nerve branches 24, 25 at a point prior to the nerves entering pudendal canals 14, 15, respectively. However, the invention is not so limited. Rather, the invention also includes embodiments in which electrodes may be implanted proximate to a portion of dorsal nerve branches 22, 23 or perineal nerve branches 24, 25 at a point after the nerves exit pudendal canals 14, 15, respectively. In additional embodiments, electrodes may apply electrical stimulation to dorsal nerve branches 22, 23 and perineal nerve branches 24, 25 via pudendal canals 14, 15, respectively. In further embodiments, electrodes may be implanted within pudendal canals 14, 15 to apply electrical stimulation to dorsal nerve branches 22, 23 and perineal nerve branches 24, 25, respectively.

The pain experienced by the patient may be unilateral or bilateral, constant or intermittent, spontaneous or exacerbated by physical activities and pressure, and may remain localized or radiate outward. A male patient, for example, may experience pain in the penis, scrotum, perineum, labia (in women) or anorectal region. Applying electrical stimulation may cause paresthesia in penis 8, scrotal skin 11, perineum, and pelvic region based on the position of the electrodes. The number and position of the leads may be dependent on the pain perceived by the patient and the type of electrical stimulation delivered to treat the pain.

In the illustrated example, IMD 4 is coupled to leads 17 and 19. Leads 17 and 19 each carry electrodes, i.e., electrodes 16 and 18, on the distal end of the lead and transmit stimulation energy from IMD 4 to electrodes 16 and 18 via conductors within leads 17 and 19 on a selective basis. In particular, one or more electrodes may be selected to form anodes and cathodes for delivery of stimulation energy via unipolar, bipolar, or multipolar electrode combinations. Each of leads 17 and 19 is shown in FIG. 1 carrying four electrodes, e.g., ring electrodes, although any number of electrodes could be used.

In some embodiments, electrodes 16 and 18 may be arranged in an axial array, e.g., as ring electrodes, or in a two-dimensional planar array, e.g., in a paddle lead. Also, other types of leads providing curved or rounded electrode arrays may be used. At least one conductor is included in each of leads 17 and 19 that electrically connects the proximal end of leads 17 and 19 to electrodes 16 and 18, respectively, in its distal end. IMD 4 may control electrical stimulation applied by each of electrodes 16 and 18 separately or control electrical stimulation by applied by a group of electrodes.

Although electrodes carried at the distal end of leads are shown in FIG. 1, the leads coupled to IMD 4 may include various types of electrodes depending on the type of stimulation delivered and the location of the lead. For example, IMD 4 may be coupled to any number and any type of electrodes, such as cuff electrodes, paddle electrodes leads, and other electrodes suitable for applying electrical stimulation to dorsal nerve branches 22, 23 and perineal nerve branches 24, 25. In addition, in some cases, leadless stimulators may be used. In any case, electrodes may be implanted at various locations proximate to dorsal branches 22, 23 and perineal branches 24, 25 of one or both pudendal nerves 20, 21 to apply stimulation uni-laterally or bi-laterally.

A cuff electrode may provide more direct electrical contact, i.e., better electrical coupling, with a dorsal or perineal nerve branch than a standard electrode lead. However, in some cases, applying electrical stimulation directly to a nerve may result in the patient experiencing an unpleasant sensation, such as a burning sensation. Consequently, a standard (non-cuff) electrode implanted proximate to the dorsal or perineal branch of the pudendal nerve may be advantageous because the patient may experience a more pleasant paresthesia as a result of stimulation. In addition, a standard ring electrode lead may also be advantageous in terms of surgical ease.

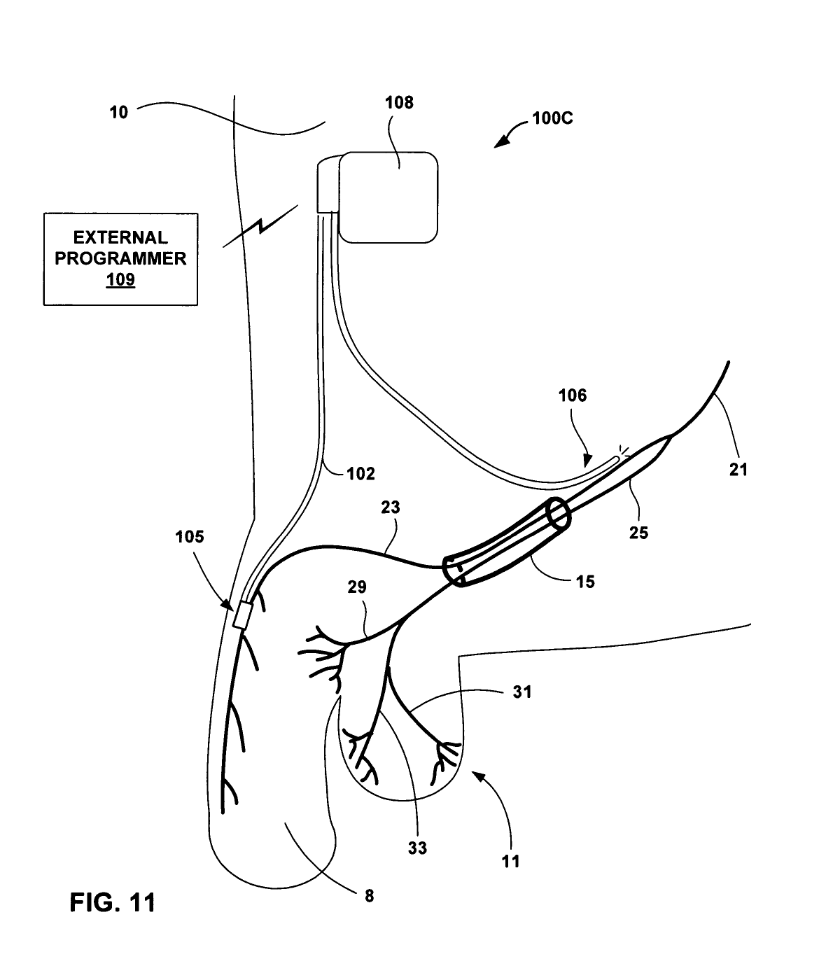

FIG. 11 illustrates an example system in which an IMD is coupled to a cuff electrode that stimulates a dorsal branch of a pudendal nerve. A cuff electrode includes a cuff-like fixation structure and one or more electrodes carried by the fixation structure. Cuff electrodes may be implanted at different locations along dorsal nerve branches 22, 23 and perineal nerve branches 24, 25.

Cuff electrodes may comprise a rigid cuff electrode, a self-sizing spiral cuff electrode, a half cuff electrode, a helical electrode, a chambered electrode, or other types of cuff electrodes that are shaped, sized and otherwise configured to at least partially wrap around a dorsal nerve branch or a perineal nerve branch. The cuff electrode may be sized and shaped to at least partially enclose a dorsal nerve branch or a perineal nerve branch and promote electrical coupling pressure between the electrode and the nerve.

Upon enclosure of at least a portion of dorsal nerve branch or a perineal nerve branch, a cuff may be held in a closed position by shape memory properties, sutures, interlocking tabs, surgical adhesive, crimping, or other fixation techniques or structures. Cuff electrodes may include a single electrode or multiple electrodes. For example, a cuff electrode may include a bipolar or multipolar arrangement of electrodes or a unipolar electrode that is referenced to the electrical potential of an active can electrode carried by IMD 4.

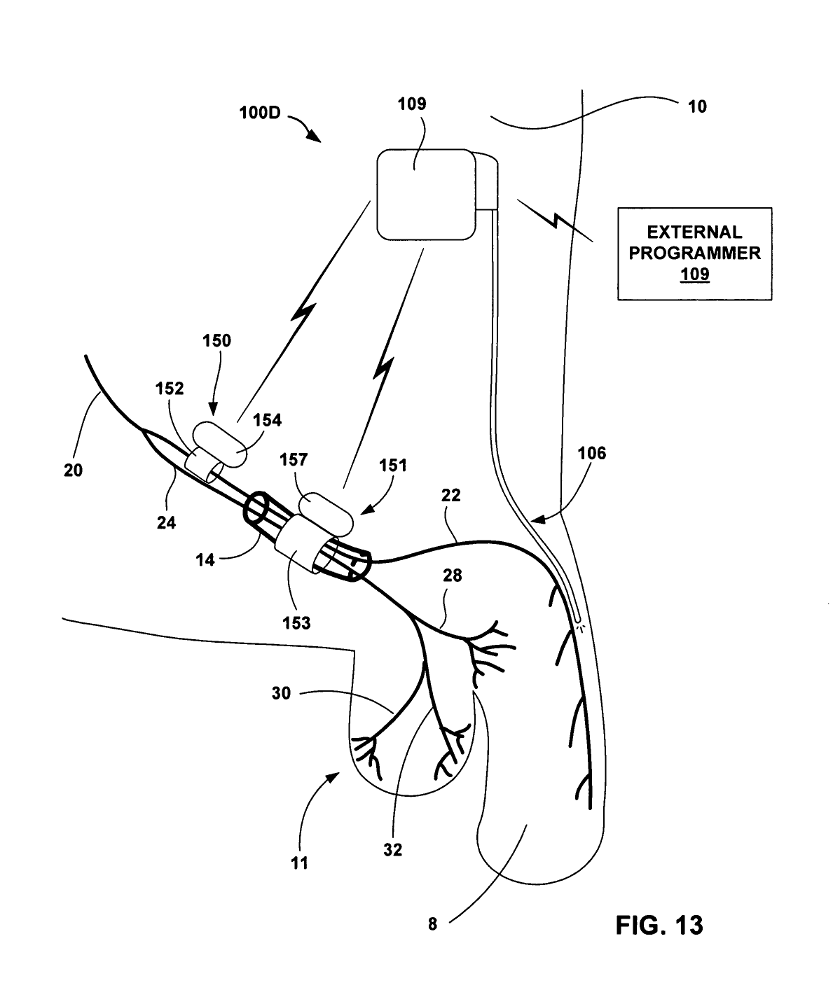

As another example, FIG. 13 illustrates leadless microstimulators that apply electrical stimulation to a dorsal and perineal nerve branch of a pudendal nerve directly and indirectly, via a pudendal canal. In this case, an IMD or external programmer may wireless control the leadless microstimulator to delivery electrical stimulation.

With further reference to FIG. 1, IMD 4 includes electrical stimulation pulse generator circuitry and delivers electrical stimulation in the form of electrical pulses in accordance with stored stimulation parameters, e.g., electrode combination, electrode polarity, pulse amplitudes, pulse widths, pulse rates, and/or duty cycle. By way of example, the electrical stimulation may include stimulation pulses having pulse widths between approximately 10 and 5000 microseconds, more preferably between approximately 100 and 1000 microseconds, and still more preferably between 180 and 450 microseconds. The stimulation pulses may have voltage amplitudes between approximately 0.1 and 50 volts, more preferably between approximately 0.5 and 20 volts, and still more preferably between approximately 1 and 10 volts. The pulses may have frequencies between approximately 0.5 and 500 hertz, more preferably between approximately 10 and 250 hertz, and still more preferably between approximately 50 and 150 hertz. The pulses may be alternating current (ac) pulses or direct current (dc) pulses, and may be mono-phasic, bi-phasic, or multi-phasic in various embodiments.

IMD 4 may drive electrodes 16 and 18 with the same or different stimulation pulses or waveforms. In some embodiments, IMD 4 may cause electrodes 16 and 18 to deliver electrical stimulation simultaneously, or in an interleaved or alternating fashion. For example, electrodes 16 and 18 may deliver electrical stimulation with different pulse rates, duty cycles or scheduled times for delivery, which may result in alternating delivery of stimulation. Interleaved or alternating delivery of stimulation may, for example, reduce the likelihood that neural accommodation or tolerance will impair the efficacy of the stimulation. Interleaved or alternating delivery of stimulation may also result in more complete pain relief than would be possible through delivery of stimulation via only one electrode or electrode array. Interleaved stimulation may be delivered by an combination of ring electrodes, paddle lead electrodes, cuff electrodes, or microstimulators.

Leads 17 and 19 may be implanted at various locations proximate to dorsal branches 22, 23 and perineal branches 24, 25 of pudendal nerves 20, 21, respectively. In the illustrated example, lead 17 is implanted proximate to a portion of dorsal nerve branch 22 prior to the nerve entering pudendal canal 14 and lead 19 is implanted proximate to a portion of perineal nerve branch 25 prior to the nerve entering pudendal canal 15, but the invention is not limited as such. Rather, leads 17 and 19 may be implanted at various locations along dorsal nerve branches 22, 23 and perineal nerve branches 24, 25.

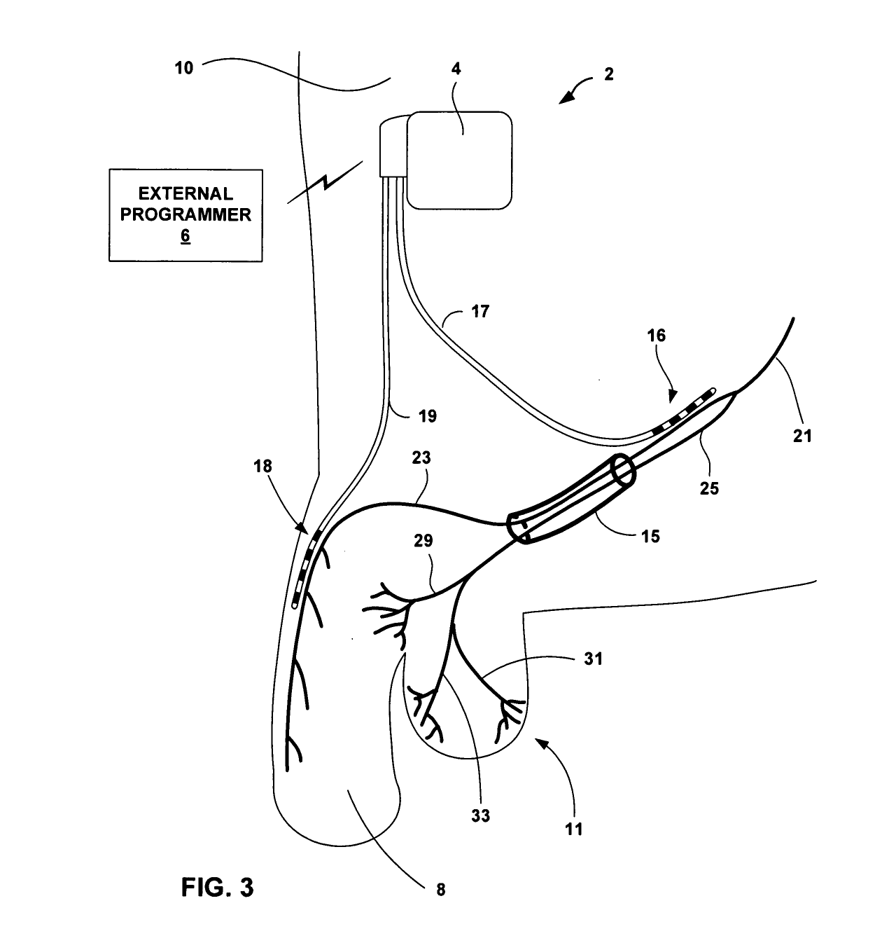

The positions of leads 17 and 19 in FIG. 1 are shown for purposes of illustration to show different possible implantation locations and associated target stimulation sites. Specifically, leads 18 and 19 illustrate two locations which may be particularly advantageous for applying electrical stimulation, which will be described in detail below. However, IMD 4 may be coupled to a single lead or a plurality of leads based on the perceived pain of the patient and his response to electrical stimulation therapy. FIGS. 3-5 illustrate alternative sites for implanting electrodes to apply electrical stimulation for pelvic disorders such as sexual dysfunction, urinary incontinence, PNE, pelvic pain, or other afflictions associated with pain originating from the pelvic or groin regions.

The following is a general anatomical description of the dorsal and perineal branches of the pudendal nerves that may be used for reference. However, the pudendal nerves and the dorsal and perineal branches of the pudendal nerves have been demonstrated to have a variable origin, course, and distribution in the pelvic region among different patients. In other words, anatomical variability may be observed from patient to patient. Accordingly, the drawings are provided as a conceptual representation to aid in the understanding of pertinent embodiments of the invention, but not necessarily as an accurate anatomical guide.

In FIG. 1, pudendal nerves 20, 21, and dorsal branches 22, 23, and perineal branches 24, 25 of pudendal nerves 20, 21 are illustrated. FIG. 1 also illustrates pudendal canals 14, 15. The pudendal nerve generally innervates the penis (in men) and clitoris (in women), bulbospongiosus and ischiocaverosus muscles, and areas around the scrotum, perineum, and anus. At sexual climax, the spasms in the bulbospongiosus and ischiocavernous result in ejaculation in the male and most of the feelings of orgasm in both sexes. These muscles pulse at approximately 0.8 Hz at orgasm in both sexes.

Although not explicitly shown in FIG. 1, pudendal nerves 20, 21 originate from the ventral branches of the second, third, and fourth sacral nerves. The pudendal nerve passes between the pififormis and coccygeus muscles (not shown) and leaves the pelvis through the lower part of the greater sciatic foramen. The pudendal nerve then crosses the spine of the ischium, and re-enters the pelvis through the lesser sciatic foramen. The pudendal nerve accompanies the internal pudendal vessels (not shown) upward and forward along the lateral wall of the ischiorectalfossa (not shown), being contained in a sheath of the pudendal canal, also termed Alcock's canal. Prior to entering the pudendal canal, the pudendal nerve divides into two terminal branches, i.e., the dorsal nerve of the penis (in men) or clitoris (in women) and the perineal nerve. Before the division into the dorsal and perineal nerve branches, the pudendal nerve gives off the inferior hemorrhoidal nerve (not shown).

The inferior hemorrhoidal nerve (not shown) occasionally arises directly from the sacral plexus (not shown) and crosses the ischiorectal fossa, with the inferior hemorrhoidal vessels (not shown), toward the anal canal (not shown) and the lower end of the rectum (not shown), and is distributed to the sphincter ani externus (not shown) and to the integument around the anus (not shown). Branches of this nerve may communicate with the perineal branch of the posterior femoral cutaneous (not shown) and with the posterior scrotal nerves at the forepart of the perineum (not shown).

The perineal nerve branch is the inferior and larger of the two terminal branches of the pudendal nerve. The perineal nerve is situated below the internal pudendal artery (not shown) and accompanies the perineal artery (not shown) and divides into a posterior scrotal branch (in men), or labial branch (in women), and a muscular branch. With reference to FIG. 1, perineal nerves 24, 25 divide into posterior scrotal branches 26, 27 and muscular branches 28, 29, respectively.

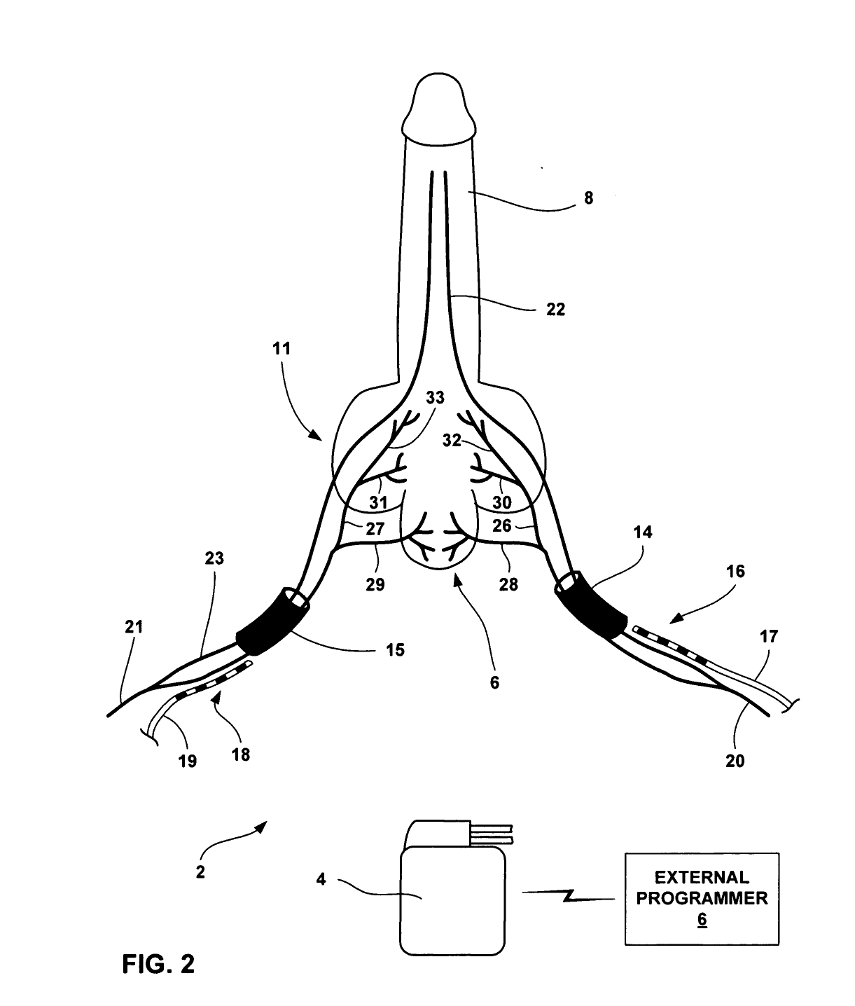

The posterior scrotal (or labial) branches are two in number, medial and lateral. The medial and lateral branches of posterior scrotal branches 26, 27 are not shown in FIG. 1. However, the medial and lateral branches of posterior scrotal branches 26, 27 are illustrated in FIGS. 2-5. The medial and lateral branches pierce the fascia of the urogenital diaphragm (not shown), and run forward along the lateral part of the urethral triangle (not shown) in company with the posterior scrotal branches (not shown) of the perineal artery (not shown). The medial and lateral branches are distributed to the skin of the scrotum and communicate with the perineal branch of the posterior femoral cutaneous nerve (not shown). These nerves supply the labium majus in females.

In FIG. 1, perineal nerves 24, 25 include muscular branches 28, 29, respectively. The muscular branches of the perineal nerve are distributed to the transverses perinaei superficialis, bulbocavernous, ischiocavernosus, and constrictor urethrae. A branch given off from the muscular branch of the perineal nerve pierces the bulbocavernosus muscle, and supplies the corpus cavernosum urethra, ending in the mucous membrane of the urethra.

The dorsal nerve of the penis is the deepest division of the pudendal nerve. The dorsal nerve accompanies the internal pudendal artery (not shown) along the ramus of the ischium (not shown) and subsequently runs forward along the margin of the inferior ramus of the pubis (not shown), between the superior and inferior layers of the fascia of the urogenital diaphragm (not shown). As the dorsal nerve pierces the inferior layer, it provides a branch to the corpus cavernosum penis, and passes forward, in combination with the dorsal artery of the penis (not shown), between the layers of the suspensory ligament (not shown), on to the dorsum of the penis, and ends on the glans penis. In the female, the dorsal nerve is typically smaller than in the male, and supplies the clitoris.

In accordance with an embodiment of the invention, electrical stimulation may be delivered via electrodes positioned proximate to a portion of at least one of dorsal branches 22, 23 or perineal branches 24, 25 of pudendal nerves 20, 21. In the illustrated example, electrodes 16 are implanted proximate to a portion of dorsal branch 22 prior to dorsal branch entering pudendal canal 14 and electrodes 18 are implanted proximate to a portion of perineal branch 25 prior to perineal branch entering pudendal canal 15.

Further, the invention includes embodiments in which electrodes are implanted proximate to a portion of a dorsal branch or a perineal branch after the nerve branch exits a pudendal canal. Implanting electrodes higher (upstream in the central nervous system), e.g., proximate to a portion of a dorsal nerve prior to the nerve entering a pudendal canal instead of proximate to a portion of a dorsal nerve after the nerve exits the pudendal canal, may result in the patient experiencing pain relief over a larger area, which may be advantageous in some instances.

With reference to a perineal branch, electrodes may be implanted proximate to a posterior scrotal branch, a muscular branch, or both. In another example, electrodes may be implanted proximate to a portion of a dorsal branch or a perineal branch within a pudendal canal. In yet another example, electrodes may indirectly apply electrical stimulation to a dorsal branch, perineal branch, or both via a pudendal canal. The invention further includes embodiments in which electrodes are implanted bi-laterally in any combination. Accordingly, the positions of electrodes 16 and 18 are merely exemplary.

Leads 17 and 19 may include fixation elements for securing electrodes 16 and 18 proximate to a portion of dorsal nerve 22, 23 and perineal nerve 25, 26, respectively. Fixation elements, such as hooks, barbs, helical structures, tissue ingrowth mechanisms, or other anchoring mechanisms may serve to fix electrodes relative to a dorsal or perineal branch of a pudendal nerve so that the electrodes can provide consistent electrical simulation. Without anchoring electrodes to a nerve branch or tissue proximate to a nerve branch, the distance between the electrodes and the nerve branch may vary as the patient moves throughout the day, reducing the efficacy of the applied electrical stimulation. However, it is possible that anchoring mechanisms may damage the dorsal branch or perineal branch of a pudendal nerve or surrounding tissue during implantation or as patient 10 moves.

Leads 17 and 19 are typically either surgically implanted or inserted percutaneously. Leads 17 and 19 may be surgically implanted using well known surgical techniques, such as the surgical procedure used for neurectomy of the pudendal nerve Prior to surgically implanting electrodes, local nerve blocks may be performed using a nerve blocking agent to determine the precise nerve involved in the pain experienced by the patient. For example, if a local nerve block in the perineal region ameliorates the patient's pain, a surgeon may conclude that electrical nerve stimulation is likely to be efficacious, and may proceed to surgically implant electrodes in accordance with the invention. Alternatively, a clinician may stimulate the patient using an insulated needle to determine the nerve involved and the placement of an electrode. The diagnosis may also be made using the results of the patient history, physical examination, and preoperative electromyography.

IMD 4 may be implanted at a site in patient 10 near dorsal branches 22, 23 and perineal branches 24, 25 of pudendal nerves 20, 21. The implantation site may be a subcutaneous location in the side of the lower abdomen. Alternatively, IMD 4 may be implanted within the scrotum or buttock of the patient. IMD 4 may be miniaturized to allow IMD 4 to be implanted within the scrotum. In any case, the surgeon may then tunnel a lead through tissue and subsequently connect the lead to IMD 4, with or without a lead extension. IMD 4 may be constructed with a biocompatible housing, such as titanium or stainless steel, much like a conventional neurostimulator such as those used for spinal cord stimulation or pelvic stimulation, e.g., for relief of chronic pain, sexual dysfunction, or urinary or fecal incontinence.

External programmer 6 may control delivery of electrical stimulation by IMD 4. For example, in some embodiments, external programmer 6 may comprise a clinician programmer or a patient programmer. A clinician programmer may be a handheld computing device including a display, such as an LCD or LED display, to display electrical stimulation parameters. A clinician programmer may also include a keypad, which may be used by a user to interact with the clinician programmer. In some embodiments, the display may be a touch screen display, and a user may interact with the clinician programmer via the display. A user may also interact with the clinician programmer using peripheral pointing devices, such as a stylus or mouse. The keypad may take the form of an alphanumeric keypad or a reduced set of keys associated with particular functions.

A clinician (not shown) may use the clinician programmer to program electrical stimulation to be delivered to patient 10. In particular, the clinician may use the clinician programmer to select values for therapy parameters, such as pulse amplitude, pulse width, pulse rate, electrode polarity and duty cycle, for one of or both electrodes 16 and 18. IMD 4 may deliver the electrical stimulation according to programs, each program including values for a plurality of such therapy parameters. In this manner, IMD 4 controls delivery of electrical stimulation according to preprogrammed stimulation programs and schedules.

When implemented as a patient programmer, external programmer 6 may be a handheld computing device. The patient programmer 26 may also include a display and a keypad to allow patient 10 to interact with the patient programmer. In some embodiments, the display may be a touch screen display, and patient 10 may interact with the patient programmer via the display. Patient 10 may also interact with the patient programmer using peripheral pointing devices, such as a stylus or mouse.

Patient 10 may use the patient programmer to control the delivery of electrical stimulation. In particular, in response to a command from patient 10, external programmer 6 may activate IMD 4 to deliver electrical stimulation or, alternatively, deactivate IMD 4 when no electrical stimulation is desired. Patient 10 may also use the patient programmer to select the programs that will be used by IMD 4 to deliver electrical stimulation. Further, patient 10 may use the patient programmer to make adjustments to programs, such as adjustments to amplitude, pulse width and/or pulse rate. Additionally, the clinician or patient 10 may use a clinician or patient programmer to create or adjust schedules for delivery of electrical stimulation.

IMD 4 and external programmer 6, implemented as a clinician programmer or a patient programmer, communicate via wireless communication. In some embodiments, external programmer 6 communicates via wireless communication with IMD 4 using radio frequency (RF) telemetry techniques known in the art. A clinician programmer and patient programmer may communicate with one another by wireless communication, e.g., to change or update programs. Alternatively, the programmers may communicate via a wired connection, such as via a serial communication cable, or via exchange of removable media, such as magnetic or optical disks, or memory cards.

As previously described, leads 17 and 19 may be implanted surgically or percutaneously. When inserted percutaneously, leads 17 and 19 may be used in conjunction with an external trial stimulator (not shown) in order to determine if permanent implantation of the electrodes and leads is an effective treatment for the patient's pain. For example, prior to implantation of IMD 4, patient 10 may engage in a trial period, in which patient 10 receives an external trial stimulator on a temporary basis. The external trial stimulator may be coupled to temporary leads or chronically implanted leads via a percutaneous lead extension.

The trial neuromodulation permits a clinician to observe neuromodulation efficacy and determine whether implantation of a chronic neuromodulation device is advisable. For example, a trial neurostimulation period may assist the clinician in selecting values for a number of programmable parameters in order to define the neurostimulation therapy delivered to patient 10. For example, the clinician may select an amplitude, which may be current- or voltage-controlled, and pulse width for a stimulation waveform to be delivered to patient 10, as well as a rate, i.e., frequency, delivered to the patient. In addition, the clinician also selects particular electrodes on a lead to be used to deliver the pulses, and the polarities of the selected electrodes.

By stimulating at least one of dorsal branches 22, 23 or perineal branches 24, 25 of pudendal nerves 20, 21, a system in accordance with an embodiment of the invention may treat pelvic disorders, such as sexual dysfunction, urinary incontinence, PNE, pelvic pain, or other afflictions associated with pain originating from the pelvic or groin regions. For example, the invention may substantially reduce or eliminate chronic pelvic pain, including urogenital pain such as chronic groin pain, chronic testicular pain (CTP), prostatitis-like pain, or pain associated with PNE without loss of sensation in the penis or scrotum or other unwanted side effects, such as sexual dysfunction and urinary incontinence.

The invention is not limited to applying electrical stimulation to treat pelvic disorders. Rather, the invention also may include embodiments in which drug therapy, i.e., delivering one or more drugs to a patient, is delivered in combination with electrical stimulation to branches of one or both pudendal nerves, e.g., dorsal branches, perineal branches, or both. Drug therapy and electrical stimulation may be delivered simultaneously or on an alternating basis.

For example, electrical stimulation may be delivered constantly or intermittently through the course of a day and the patient may use a patient programmer to deliver drug therapy when experiencing moments of increased pain. Alternatively, drug therapy may be delivered according to preprogrammed parameter sets and schedules and the patient may use a patient programmer to deliver electrical stimulation when the drug therapy does not substantially reduce the pain. In either case, the combined delivery of drug therapy and electrical stimulation and one or more drugs supports neuromodulation therapy to alleviate pain or other symptoms associated with pelvic region disorders.

In some embodiments, system 2 includes an implantable drug delivery device that delivers one or more drugs to at least one branch of one or both pudendal nerves in combination with the previously described electrical stimulation. Such systems deliver drugs to at least one of dorsal branches 22, 23 or perineal branches 24, 25 of pudendal nerves 20, 21 via fluid transfer devices. Fluid transfer devices may comprise a catheter, a conduit, or the like, that enables the transfer of fluid from the implanted drug delivery device to the delivery site. Accordingly, a fluid transfer device may be implanted at various locations along dorsal branches 22, 23 or perineal nerves 24, 25 in the same manner as electrodes that apply electrical stimulation.

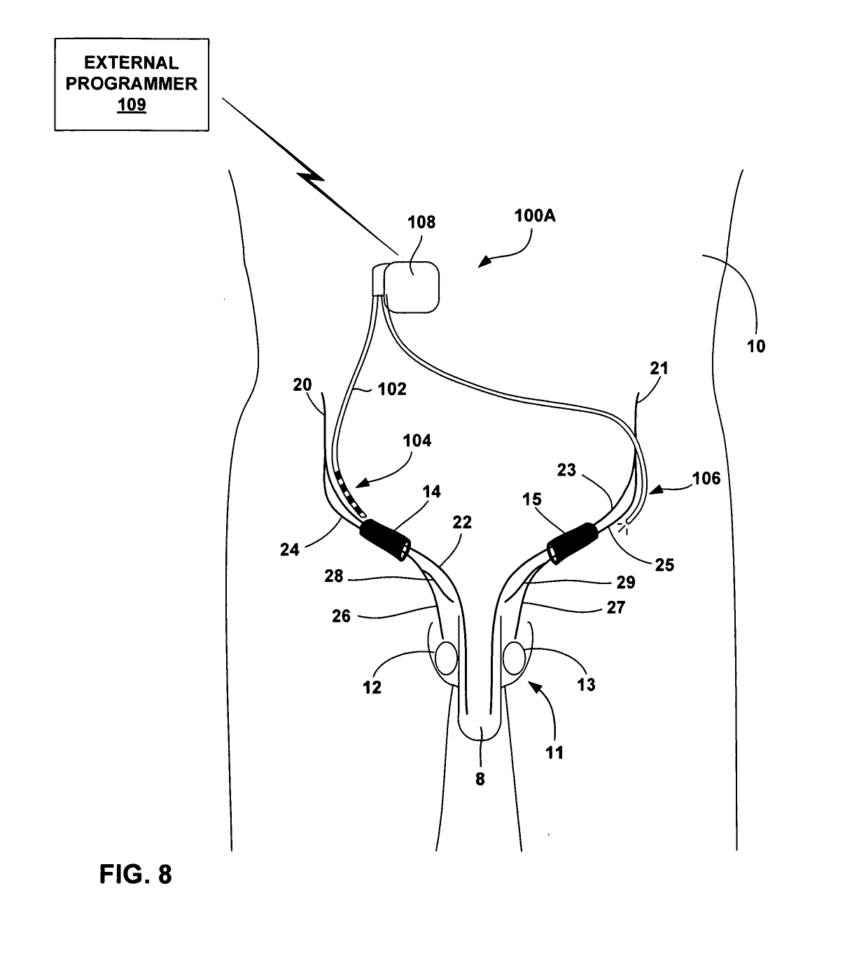

FIGS. 8, 9, 11, and 13 illustrate example systems that include an IMD for delivering drug therapy in combination with electrical stimulation to at least one of dorsal branches 22, 23 or perineal branches 24, 25 of patient 10 to treat a pelvic disorder such as sexual dysfunction, urinary incontinence, pudendal nerve entrapment (PNE), chronic testicular pain (CTP), chronic groin pain, prostatitis-like pain, and urogenital pain or other forms of pelvic pain that cause long term (chronic) pain in the pelvic or groin region. However, the invention is not limited to the embodiments shown in FIGS. 8, 9, 11, and 13. Rather, FIGS. 8, 9, 11, and 13 illustrate example embodiments showing different locations at which fluid transfer devices may be implanted and in which drug therapy is delivered in combination with electrical stimulation via various types of electrodes. Fluid transfer devices, in general, may be implanted proximate to dorsal branches 22, 23 or perineal branches 24, 25 at any location as previously described with respect to electrodes. Such embodiments include embodiments in which fluid transfer devices are implanted uni-laterally or bi-laterally in any combination without listing exhaustively listing all possible combinations. Accordingly, the positions of fluid transfer devices in FIGS. 8, 9, 11, and 13 are merely exemplary.

The fluid transfer devices may be coupled to an implantable drug delivery device implanted within a subcutaneous pocket in the abdomen of the patient or, alternatively, the scrotum or buttock of the patient. The implantable drug delivery device may be incorporated within IMD 4 or may be independent of IMD 4.

The implanted drug delivery device may include one or more reservoirs. Each reservoir may contain a drug or a mixture of drugs. By way of example, and without limitation, IMD 4 may contain one or more of a variety of drugs. In general, such a drug may be selected to treat sexual dysfunction, urinary incontinence, or alleviate chronic pelvic pain, including urogenital pain such as chronic groin pain, chronic testicular pain (CTP), urogenital pain, prostatitis-like pain, or pain associated with PNE.

In pain applications, for example, the IMD 4 may deliver one or more of a variety of drugs such as gabapentin, morphine, clonidine, tizanidine, hydromorphone, fentanyl, sufentanil, methadone, meperidine, tetracaine, bupivicaine, zinconotide, adenosine, ketorolac, baclofen, ropivicaine, ketamine, octreotide, neostigmine, or droperidol. For incontinence therapy, the IMD 4 may deliver one or more of the following: Ditropan (Oxybutynin chloride) or Detrol (tolterodine tartrate), which both treat symptoms of overactive bladder, including frequent urination, urgency, and urge incontinence, by blocking the nerve impulses that prompt the bladder to contract. For sexual dysfunction therapy, the IMD 4 may deliver one or more of the following: Cialis (tadalafil), Levitra (vardenafil), Viagra (sildenafil citrate), which work to dilate blood vessels in the penis, allowing inflow of blood need to achieve and maintain an erection. Other drugs or agents for delivery by IMD 4 for sexual dysfunction therapy may include hormones such as estrogen or testosterone, the L-arginine amino acid, prostglandin E1, phentomlamine (Vasomax), apomorphine, yohimbine, phentolamine, thymoxamine, papaverine, verapamil, imipramine, guanethidine, and metaraminol.

In some embodiments, each fluid transfer device may be coupled to the same reservoir or different reservoirs. The implantable drug delivery device also may include one or more pumps that deliver drugs from the reservoirs to the fluid transfer devices. The implanted drug delivery device may control which drugs and the dosage and rate at which the drugs are delivered by controlling which pumps are active. The drug delivery device may be programmed prior to implanting the drug delivery device within the patient or, alternatively, programmed via external programmer 6. A clinician programmer may use external programmer 6 to program a drug delivery method for patient 10. For example, the drugs may be delivered by a constant drip, a periodic bolus, a combination of these methods, or another delivery method. The present invention is not limited to a particular drug delivery method.

In addition to programming electrical stimulation for patient 10, a clinician or patient 10 may also use external programmer 6 to program drug delivery to patient 10. In particular, the clinician or patient may operate external programmer 6 to adjust delivery parameters, such as which of the plurality of drugs contained in the device are delivered and the dosage and rate at which the drugs delivered. In some cases, the clinician or patient 10 may use external programmer 6 to activate the drug delivery device to deliver drugs or, alternatively, deactivate the drug delivery device when no drugs are desired. Patient programmer 29, drug delivery device, or both may apply maximum dosage rate limits, and lockout intervals, to prevent delivery of excessive amounts of the drug in response to patient requests. Patient 10 may also use external programmer 6 to select the programs that will be used by drug delivery device to deliver the drugs. Further, patient 10 may use external programmer 6 to create or adjust schedules for delivery of drugs.

FIG. 2 is a schematic diagram further illustrating system 2. In particular, system 2 is illustrated from a top view of male patient 10. For purposes of illustration, only pudendal nerves 20, 21, dorsal branches 22, 23 and perineal branches 24, 25 of pudendal nerves 20, 21, pudendal canals 14, 15, penis 8, and scrotum 11 are shown. FIG. 2 illustrates pudendal nerves 20, 21 branching to form dorsal branches 22, 23 and perineal branches 24, 25 branches, respectively, prior to dorsal branches 22, 23 and perineal branches 24, 25 entering pudendal canal 14, 15, respectively. However, in some patients, the branch point of pudendal nerves 20, 21 may be located within pudendal canals 14, 15, respectively. Additionally, FIG. 2 illustrates posterior scrotal branches 26, 27 innervating scrotum 11 and muscular branches 28, 29 of perineal nerves 24, 25 innervating perineum 6, respectively. FIG. 2 also illustrates medial posterior scrotal branches 30, 31 and lateral posterior scrotal branches 32, 33 of posterior scrotal branches 26, 27.

In general, leads 17 and 19 may include fixation means such as sutures or anchoring devices that enable electrodes 16 and 18 to remain in place as patient 10 moves. However, such fixation means may damage tissue or the nerve itself, possibly causing additional pain which may reduce the efficacy of the electrical stimulation. Consequently, electrodes 16 and 18 may be implanted proximate to dorsal branch 22 and perineal branch 25, respectively by fixing leads 17 and 19 to tissue adjacent to dorsal branch 22 and perineal branch 25 via fixation means.

Although leads 17 and 19 are illustrated in FIG. 2 carrying electrodes 16 and 18, e.g., ring electrodes, leads 17 and 19 may include various types of electrodes depending on the type of stimulation delivered and the location of the lead. For example, IMD 4 may be coupled to any number and any type of electrodes, such as cuff electrodes, paddle electrodes leads, and other electrodes suitable for applying electrical stimulation to dorsal nerve branches 22, 23 and perineal nerve branches 24, 25. In addition, in some cases, leadless stimulators may be used. In any case, electrodes may be implanted at various locations proximate to dorsal branches 22, 23 and perineal branches 24, 25 of one or both pudendal nerves 20, 21 to apply stimulation uni-laterally or bi-laterally. FIGS. 3-5, 11, and 13 illustrate embodiments with various types and configurations of electrodes.

Again, system may also include an implantable drug delivery device that delivers one or more drugs, i.e., drug therapy, to at least one of dorsal branches 22, 23 or perineal branches 24, 25 in combination with electrical stimulation. For example, FIGS. 8, 9, 11, and 13 illustrate an example system that includes an IMD for delivering drug therapy in combination with electrical stimulation. Such systems include one or more fluid transfer devices that deliver a drug from the drug delivery device to the target site to treat pelvic disorders such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region

FIG. 3 is a schematic diagram further illustrating system 2. In particular, system 2 is illustrated from the left side of a male patient 10. For purposes of illustration, FIG. 3 illustrates pudendal nerve 21, dorsal branch 23 and perineal branch 25 of pudendal nerve 21, pudendal canal 15, penis 8, scrotum 11, perineum 6, posterior scrotal branch 27 and muscular branch 29 of perineal nerve 25, and medial posterior scrotal branch 31 and lateral posterior scrotal branch 33 of posterior scrotal branch 27.

In particular, FIG. 3 illustrates an embodiment in which multiple electrodes are implanted along dorsal branch 23 of pudendal nerve 21. Accordingly, similar to FIG. 2, electrode 16 is illustrated as being implanted proximate to dorsal branch 23 at a point prior to dorsal branch 23 entering pudendal canal 15 in FIG. 3. However, in FIG. 3, electrode 18 is shown as being implanted at a point after dorsal branch 23 exits pudendal canal 15 to illustrate another one of the various locations at which electrodes may be implanted. Because electrodes 16 are implanted higher (upstream in the central nervous system) from electrodes 18, patient 10 may experience pain relief over a larger area, which may be advantageous in some instances.

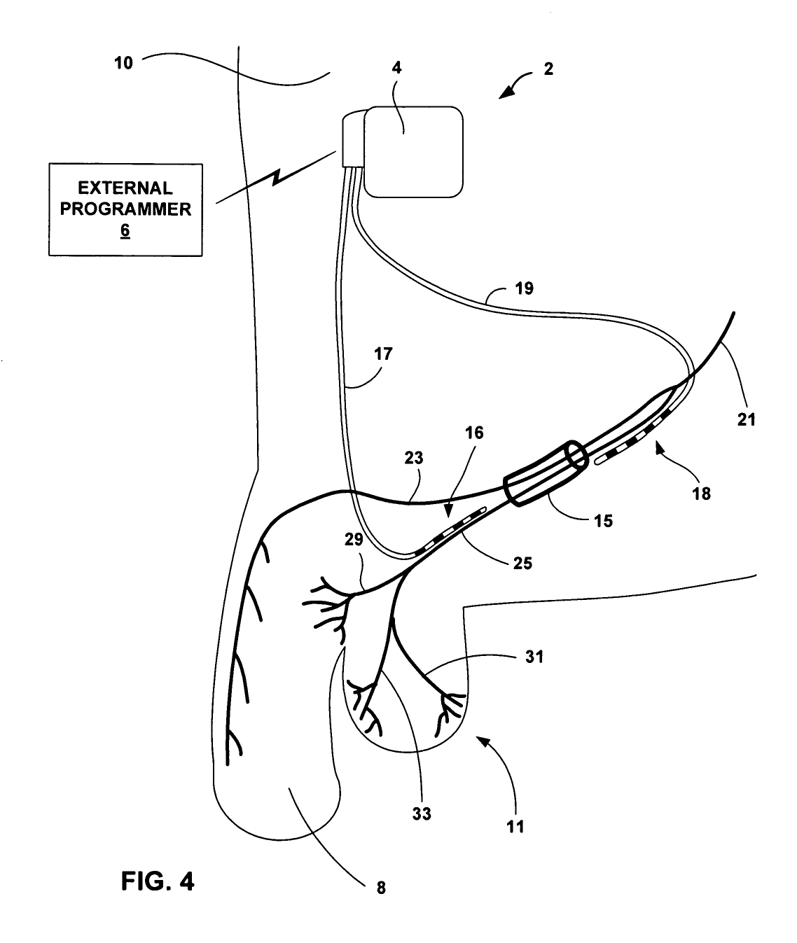

FIG. 4 is another schematic diagram further illustrating system 2 from the left side of male patient 10. Again, similar to FIG. 3, FIG. 4 illustrates pudendal nerve 21, dorsal branch 23 and perineal branch 25 of pudendal nerve 21, pudendal canal 15, penis 8, scrotum 11, perineum 6, posterior scrotal branch 27 and muscular branch 29 of perineal nerve 25, and medial posterior scrotal branch 31 and lateral posterior scrotal branch 33 of posterior scrotal branch 27 for purposes of illustration.

FIG. 4 illustrates an embodiment in which multiple electrodes are implanted along perineal branch 25 of pudendal nerve 21. Accordingly, similar to FIG. 1, electrodes 18 at distal end of lead 19 are illustrated as being implanted proximate to perineal branch 25 at a point prior to entering pudendal canal 15 in FIG. 3. However, in FIG. 3, electrodes 16 on lead 17 are shown as being implanted proximate to perineal branch 25 at a point after perineal branch 25 exits pudendal canal 15 to illustrate another one of the various locations at which electrodes may be implanted. Because electrodes 18 are implanted higher (upstream in the central nervous system) from electrodes 16, patient 10 may experience pain relief over a larger area, which may be advantageous in some instances.

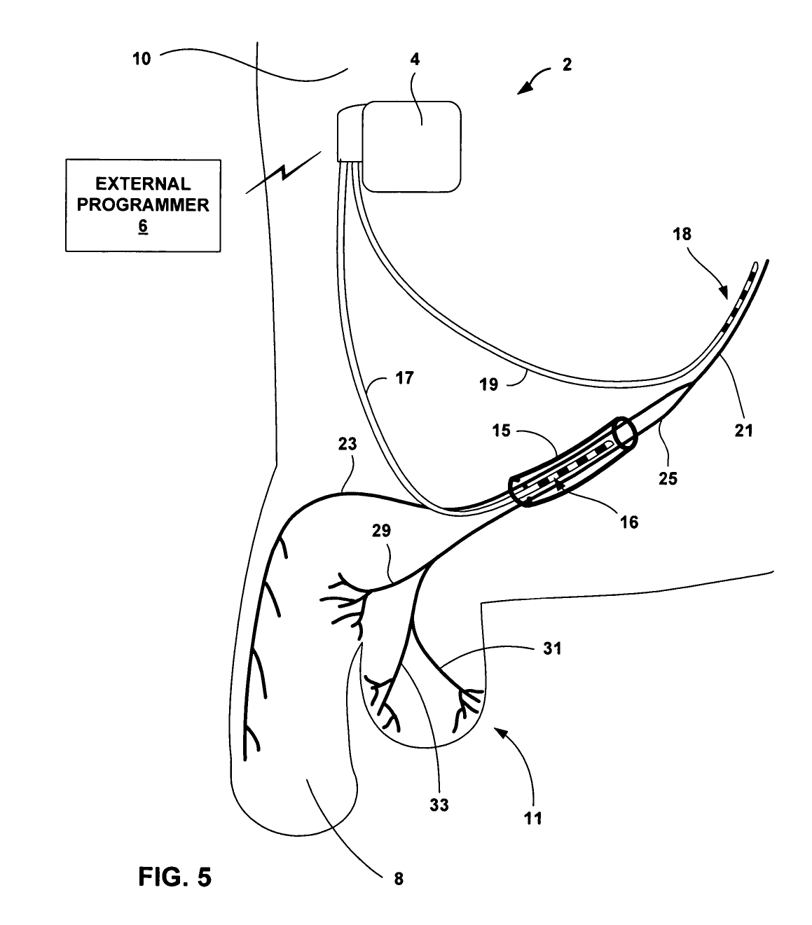

FIG. 5 is another schematic diagram further illustrating system 2 from the left side of male patient 10. Similar to FIGS. 3 and 4, FIG. 5 illustrates pudendal nerve 21, dorsal branch 23 and perineal branch 25 of pudendal nerve 21, pudendal canal 15, penis 8, scrotum 11, perineum 6, posterior scrotal branch 27 and muscular branch 29 of perineal nerve 25, and medial posterior scrotal branch 31 and lateral posterior scrotal branch 33 of posterior scrotal branch 27 for purposes of illustration.

FIG. 5 illustrates another configuration of system 2. In the example of FIG. 5, electrodes 16 are implanted proximate to dorsal nerve branch 23 and perineal nerve branch 25 of pudendal nerve 21 within pudendal canal 15 and electrodes 18 are implanted proximate to pudendal nerve 21 before it branches to form dorsal branch 23 and perineal branch 25. Although FIG. 5 shows electrodes 16 implanted approximately equally proximate to dorsal branch 23 and perineal branch 25, it may be possible to implant electrodes within a pudendal canal proximate to only one of the branches of the pudendal nerve. In this case, stimulation may be substantially applied to only one of the dorsal branch and the perineal branch. When the pudendal canal is sufficiently small in size, however, electrodes implanted within the pudendal canal may stimulate both branches of the pudendal nerve. In either case, the electrical stimulation may treat a pelvic disorder such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region.

Electrodes 16 may alternatively apply electrical stimulation to one or both of dorsal branch 23 and perineal branch 25 via pudendal canal 15. In this case, electrodes 16 may be implanted proximate to the outer fascia of pudendal, e.g., by securing lead 17 to tissue proximate to pudendal canal 15, or to the outer fascia of pudendal canal 15. The external fascia may serve to protect dorsal branch 23 and perineal branch 25 from being damaged, e.g., from pinching, stretching, lesions, or other damage, when electrodes 16 are implanted. In particular, the fascia prevents electrodes 16 from being in direct contact with dorsal branch 23, perineal branch 25, or both, which may result in a more pleasant paresthesia because electrical stimulation is delivered to one or both branches indirectly.

FIG. 5 also illustrates electrodes 18 carried at the distal end of lead 19 implanted proximate to pudendal nerve 18 above its branch point where dorsal branch 23 and perineal branch 25 originate. Implanting electrodes 18 proximate to pudendal nerve 21 in this manner may be particularly advantageous when electrodes, or drug therapy delivered in combination with electrical stimulation, apply electrical stimulation at another location along dorsal branch 23, perineal branch 25, or both. For example, when the electrical stimulation applied by electrodes 16, at the illustrated location or other locations in accordance with embodiments of the invention, does not sufficiently treat the pelvic disorder affecting patient 10, e.g., pain associated with PNE, electrodes 18 may apply additional electrical stimulation that results in patient 10 experiencing more complete relief from pain.

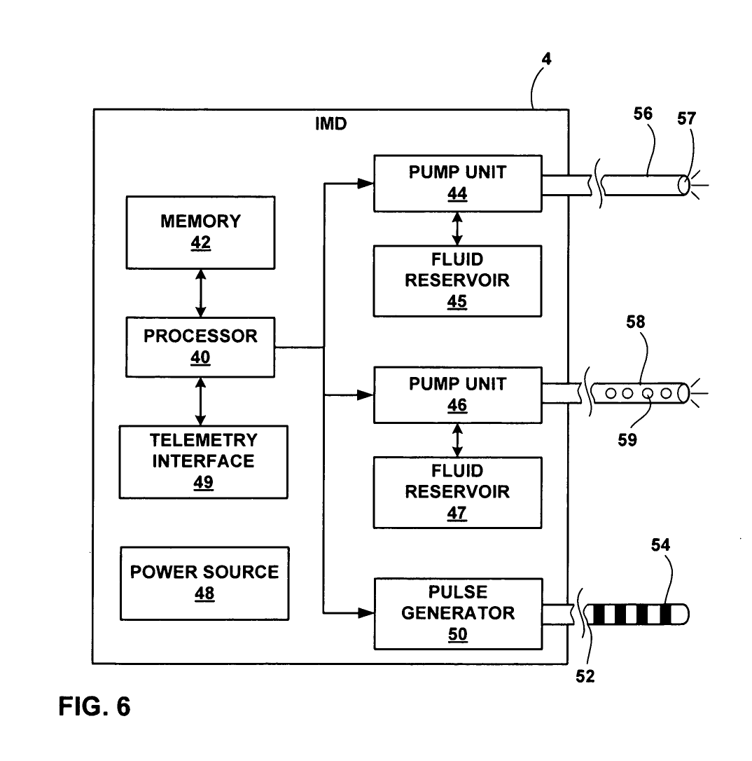

FIG. 6 is a block diagram illustrating an example configuration of IMD 4. IMD 4 may apply electrical stimulation at least one of dorsal nerve branches 22, 23 or perineal branches 24, 25 of pudendal nerves 20, 21, respectively, via electrodes, e.g., electrodes 16 and 18 carried at the distal ends of leads 17 and 19. In some embodiments, however, a drug delivery device may also deliver one or more drugs in combination with electrical stimulation to stimulation at least one of dorsal nerve branches 22, 23 or perineal branches 24, 25 via one or more fluid transfer devices. In embodiments in which drug therapy is delivered to at least one of dorsal nerve branches 22, 23 or perineal branches 24, 25 in combination with electrical stimulation, the drug delivery device may be incorporated with the electrical stimulation device or the drug delivery device and electrical stimulation device may be independent of each other, i.e., contained within separate housings. In the illustrated example of FIG. 6, IMD 4 incorporates the electrical stimulation device with the drug delivery device in a common housing.

By incorporating the drug delivery device and electrical stimulation device in a common housing of an IMD, circuitry associated with both devices, such as a processor and memory, may be shared and fabricated on a single circuit board. As a result, the IMD may be substantially smaller in size and cost less than separate drug delivery and electrical stimulation devices. Additionally, the IMD may be implanted within the patient using fewer incisions and requiring less space than separately implanting drug delivery and electrical stimulation devices.

In FIG. 6, IMD 4 is illustrated having fluid transfer devices 16 and 18 for delivering drug therapy and one or more electrodes 54, carried by one or more implantable leads 52, for delivering electrical stimulation to a patient. The configuration, type, and number of fluid transfer devices and electrodes in FIG. 6 are merely exemplary. In addition to, or in place of ring electrodes 54, IMD 4 may include any number and any type of electrodes, such as cuff, paddle electrode leads, and leadless stimulators. A leadless stimulator does not generally include any elongated leads, and instead carries electrodes on a housing of the stimulator or on a structure such as a fixation device extending from the housing.

Each fluid transfer device, e.g., a catheter, may have an elongated, tubular body with an inner lumen. With reference to FIG. 6, the body may include a proximal opening to receive the drug, and a distal opening 57 for delivery of the drug to a target site. Additionally, or alternatively, the elongated body may include a series of lateral outlets 59 formed in a lateral wall of the body. The outlets provide fluid communication between the inner lumen and the outside of the elongated body. The outlets 59 may be positioned at various axial positions along the length of the elongated body, as well as at various circumferential positions. The lateral outlets may be concentrated toward a distal end of the fluid transfer device.

In the example of FIG. 6, IMD 4 delivers one or more drugs to at least one of dorsal nerve branches or perineal branches of a patient via fluid transfer devices 56 and 58 to alleviate a pelvic disorder such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region. Fluid transfer devices 56, 58 may be coupled to a common fluid reservoir and pump unit, or separate fluid reservoirs 45, 47 and pump units 44, 46. IMD 4 may also apply electrical stimulation to one or more branches of one or both pudendal nerves of the patient via electrodes 54 in combination with the drug therapy. IMD 4 includes a processor 40, which may take the form of one or more microprocessors, digital signal processors (DSPs), application specific integrated circuits (ASICs), field-programmable gate arrays (FPGAs), other discrete or integrated logic circuitry, or any combination of such components. IMD 4 also includes pump unit 44, pump unit 46, and pulse generator 50 which operate under the control of processor 40 to deliver drugs and electrical stimulation to the patient.

In the example of FIG. 6, fluid transfer devices 56 and 58 are coupled to fluid reservoirs 45 and 47 via pump units 44 and 46, respectively. In some embodiments of the present invention, each fluid transfer device may be coupled to more than one reservoir, or more than one fluid transfer device may be coupled to a common reservoir. If fluid transfer devices 56, 58 are coupled to the same reservoir and pump unit, each fluid transfer device may simultaneously deliver the drug to respective target sites. Alternatively, if fluid transfer devices 56, 58 are intended to deliver the drug at different times, separate pump units or a valve coupled to a common pump unit may be provided to control flow to the fluid transfer devices.

Each of fluid reservoirs 45 and 47 may contain a drug or a mixture of drugs or other fluids or agents. Pump units 44 and 46 pump the drugs from fluid reservoirs 45 and 47 to the target site via fluid transfer devices 56 and 58, respectively. Fluid reservoirs 45 and 47 may provide access for filling, e.g., by percutaneous injection of fluid via a self-sealing injection port. Fluid transfer devices 56 and 58 may comprise, for example, catheters that deliver, i.e., infuse or disperse, drugs from fluid reservoirs 45 and 47 to the same or different target sites along at least one branch of a pudendal nerve, i.e., a dorsal branch or a perineal branch.

The target site may depend on the drug being delivered. Each of fluid transfer devices 56 and 58 may dispense drugs at one or more target sties. For example, one or both of fluid transfer devices 56 and 58 may deliver drugs to one or both of dorsal and perineal branches of a pudendal nerve. The invention further includes embodiments in which fluid transfer devices are implanted in any combination uni-laterally or bi-laterally. In some embodiments, fluid transfer devices 56 and 58 need not deliver drugs to the same target site.

Processor 40 controls delivery of drug therapy according to a selected parameter set stored in memory 42. Specifically, processor 40 may control pump units 44 and 46 to deliver drug therapy with a drug contained in IMD 4 and the dosage of the drug specified by the programs of the selected parameter set. For example, processor 40 may control which drugs are delivered by IMD 4 by controlling which of pump units 44 and 46 are active. Processor 40 may also control the dosage of the drugs delivered by IMD 4 by controlling the activity of pump units 44 and 46. Processor 40 may control each of pump units 44 and 46 to deliver drug therapy according to a different program of the parameter set. The drugs may be delivered by a constant drip, a periodic bolus, a combination of these methods, or some other delivery method. The invention is not limited to a particular drug delivery method.

Processor 40 may also control pulse generator circuit 50 to deliver electrical stimulation pulses with the amplitudes and widths, and at the rates specified by the programs of the selected parameter set. Processor 40 may also control pulse generator circuit 50 to deliver each pulse according to a different program of the parameter set.

Memory 42 may store parameter sets that are available to be selected by patient 10 for delivery of drug therapy and, in some embodiments, electrical stimulation. Memory 42 may also store schedules. Memory 42 may include any combination of volatile, non-volatile, removable, magnetic, optical, or solid state media, such as read-only memory (ROM), random access memory (RAM), electronically-erasable programmable ROM (EEPROM), flash memory, or the like.

IMD 4 delivers drugs according to preprogrammed stimulation parameters and, optionally, schedules stored in memory 42. Schedules may define times for processor 40 to select particular parameter sets and control pump units 44 and 46 and pulse generator circuit 50 according to that parameter set. A schedule may cause pump units 44 and 46 to deliver drugs from fluid reservoirs 45 and 47 at respective times, which may include simultaneous and/or alternate delivery. For example, stimulation may be activated, deactivated, or altered at different times of the day, such as times during which the patient is awake or sleeping, or working or at rest. In addition, a schedule may deliver electrical stimulation in combination with drug therapy on a simultaneous or alternating basis. A clinician may create, modify, and select schedules from memory 42 using external programmer 6.

In the illustrated example of FIG. 6, electrodes 54 are electrically coupled to pulse generator 50 via conductors within lead 52. In general, IMD 4 may include any number and type of electrodes. However, a greater or lesser number of electrodes may be coupled to IMD 4 to deliver electrical stimulation to patient 10. In some embodiments, a cuff electrode may provide more direct electrical contact, i.e., better electrical coupling, with a dorsal branch, perineal branch, or pudendal canal than a standard ring electrode lead. However, in some cases, applying electrical stimulation directly to a nerve may result in the patient experiencing an unpleasant sensation, such as a burning sensation. Consequently, a standard ring electrode implanted proximate to a dorsal branch, perineal branch, or pudendal canal may be advantageous because the patient may experience a more pleasant paresthesia as a result of stimulation. In addition, a standard ring electrode lead may also be advantageous in terms of surgical ease.

FIGS. 11 and 13 illustrate various configurations with different types and numbers of electrodes. In general, a relatively large number of electrodes, e.g., from eight to thirty-two, may be desirable in order to permit selection of a greater number of bipolar, multipolar, and unipolar electrode combinations to deliver electrical stimulation. The availability of multiple, selectable electrode combinations increases the probability that an efficacious electrode combination can be selected.

Pulse generator 50 may comprise circuitry, such as capacitors and switches, for the generation of electrical stimulation in the form of pulses. In some embodiments, pulse generator circuit 50 may also include a switch device or switch matrix for selecting one or more electrode for delivery of generated stimulation pulses. Accordingly, processor 40 may select one or more electrodes and the polarities of each of the selected electrodes to deliver electrical stimulation to the patient. Under control of processor 40, pulse generator circuit 50 delivers the pulses to the selected electrodes via wires of lead 52 that are electrically connected to pulse generator 50. For example, as mentioned above, pulse generator 50 may include a switch device that switches stimulation pulses across selected electrodes.

IMD 4 also includes a wireless telemetry circuit 49 that allows processor 40 to communicate with external programmer 6, i.e., a clinician programmer or patient programmer. Processor 40 may receive programs to test on patient 10 from external programmer 6 via telemetry circuit 49 during programming by a clinician. Where IMD 4 stores parameter sets in memory 42, processor 40 may receive parameter sets from external programmer 6 via telemetry circuit 49 during programming by a clinician, and later receive parameter set selections made by patient 10 from external programmer 6 via telemetry circuit 49. Where external programmer 6 stores the parameter sets, processor 40 may receive parameter sets selected by patient 10 from external programmer 6 via telemetry circuit 49. In addition, processor 40 may receive parameter adjustments form external programmer 6.

The illustrated components of IMD 4 receive energy from a power source 48, such as a battery or other suitable power source. In some embodiments, power source 48 may be rechargeable and receives energy inductively captured by a recharge module (not shown). Power management circuitry (not shown) may control the recharging and discharging of power source 48. In other embodiments, power source 48 includes a nonrechargeable battery. In additional embodiments, power source 48 may receive operating power by inductive energy transfer with an external power source.

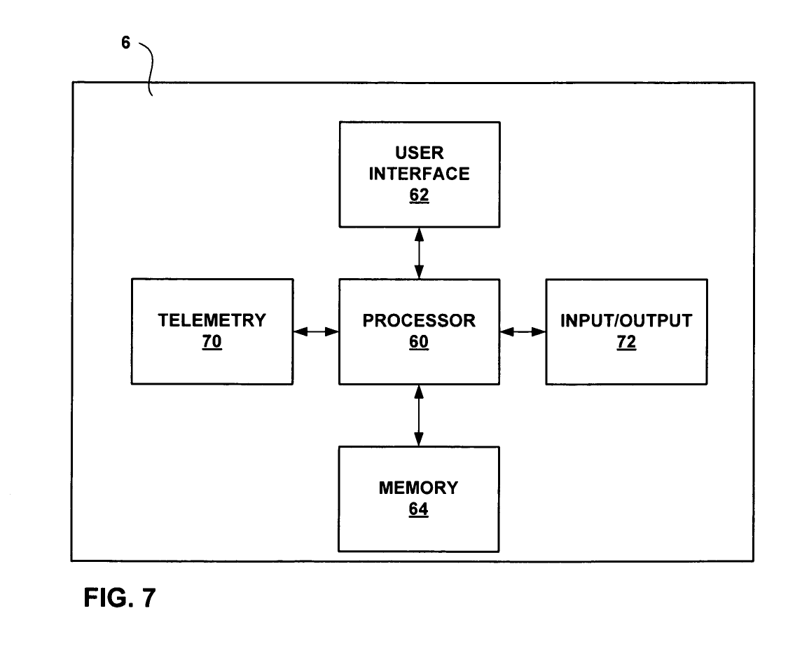

FIG. 7 is a block diagram illustrating an example patient or clinician programmer 6 that allows a patient or clinician to program drug therapy and, in some embodiments, electrical stimulation in combination with drug therapy to at least one of a dorsal branch or a perineal branch of one or both pudendal nerves of a patient. Patient 10 or a clinician may interact with a processor 60 via a user interface 62 in order to control delivery of drug therapy and electrical stimulation as described herein. User interface 62 may include a display and a keypad, and may also include a touch screen or peripheral pointing devices as described above. Processor 60 may also provide a graphical user interface (GUI) to facilitate interaction with patient 10, as will be described in greater detail below. Processor 60 may include a microprocessor, a controller, a DSP, an ASIC, an FPGA, discrete logic circuitry, or the like.

Programmer 6 also includes a memory 64. In some embodiments, memory 64 may store parameter sets that are available to be selected by patient 10 or a clinician for delivery of drug therapy and electrical stimulation. Memory 64 may also store schedules. Hence, parameter sets and schedules may be stored in IMD 4, patient programmer 6, or both. Programmer 6 also includes a telemetry circuit 70 that allows processor 60 to communicate with IMD 4, and, optionally, input/output circuitry 72 that allow processor 60 to communicate with another programmer.

Processor 60 may receive parameter set selections made by patient 10 or a clinician via user interface 62, and may either transmit the selection or the selected parameter set to IMD 4 via telemetry circuitry 70 for delivery of drug therapy and electrical stimulation according to the selected parameter set. Where patient programmer 6 stores parameter sets 66 in memory 64, processor 60 may receive parameter sets 66 from another programmer via input/output circuitry 72 during programming by a clinician. For example, a patient programmer may receive parameter sets from a clinician programmer. Circuitry 72 may include a transceiver for wireless communication, appropriate ports for wired communication or communication via removable electrical media, or appropriate drives for communication via removable magnetic or optical media. If wireless communication is used, telemetry circuitry 70 may support both wireless communication with IMD 4 and wireless communication with another programmer.

FIG. 8 is a schematic diagram illustrating an example system 100A for delivery of electrical stimulation in combination with one or more drugs to a male patient 10 to treat a pelvic disorder such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region. System 100A also may be useful for alleviation of pelvic pain or treatment of other disorders in female patients. In the illustrated example, system 100A includes electrodes 104 deployed on a lead 102 extending from an IMD 108, and a fluid transfer device 106 coupled to IMD 108. Electrodes 104 and fluid transfer device 106 deliver electrical stimulation and drug therapy to dorsal branch 22 of pudendal nerve 20 and perineal branch 25 of pudendal nerve 21, respectively, and illustrate an exemplary arrangement for delivering electrical stimulation in combination with drug therapy. However, the invention is not limited to the illustrated example. Rather, stimulation energy may be delivered to one or more of dorsal branches 22, 23 and perineal branches 24, 25 via any combination of electrodes, including axial electrode arrays, planar electrode arrays (e.g., on paddle lead), leadless microstimulators, cuff electrodes or other types of electrodes. In a similar manner drug therapy may be delivered to one or more of dorsal branches 22, 23 and perineal branches 24, via fluid transfer devices coupled to IMD 108.

IMD 108 controls the delivery of drug therapy and electrical stimulation according to preprogrammed programs, parameter sets and/or schedules. In particular, external programmer 109 may wirelessly control IMD 108 to deliver one or more drugs to at least one of dorsal branches 22, 23 and perineal branches 24, 25 via fluid transfer device 106. In the example of FIG. 8, IMD 108 is also coupled to electrodes 104 via lead 102 that apply electrical stimulation to dorsal branch 22 under the control of IMD 108. Again, the invention is not limited to the illustrated configuration. In general, IMD 108 may be coupled to any number and type of fluid transfer devices and electrodes. The fluid transfer devices and electrodes may be positioned adjacent to dorsal branches and perineal branches of one or both pudendal nerves of a patient based on the perceived pain of the patient. However, FIG. 8 merely illustrates example system 100A in which fluid transfer device 106 and electrodes 104 deliver bi-lateral drug therapy and electrical stimulation to dorsal branch 22 and perineal branch 25 of pudendal nerves 20, 21, respectively.

In the illustrated example, fluid transfer device 106 is implanted adjacent to perineal branch 25 and delivers a drug or mixture of drugs contained within IMD 108 to patient 10. As previously described, fluid transfer device 106 may include fixation elements for securing fluid transfer device 106 to tissue adjacent to perineal branch 25. Fixation elements may assist in keeping fluid transfer device 106 in close proximity to perineal branch 25 as patient 10 moves. Without fixation elements, the distance between fluid transfer device 106 and perineal branch 25 may vary through the day reducing the efficacy of the drug therapy. Fixation elements may comprise hooks, barbs, helical ingrowth devices, or other anchoring devices. Direct contact of fluid transfer device 106 and, more particularly, fixation elements with perineal branch 25 may be undesirable because direct contact may damage perineal branch 25 as patient 10 moves or if fluid transfer device 106 is removed.

The position of fluid transfer device 106 in FIG. 8 is for purposes of illustration. In practice, fluid transfer device 106 may be implanted proximate to perineal branch 25 at a point after perineal branch 25 exits pudendal canal 15. However, delivering drug therapy at a higher position along perineal branch 25 (upstream in the CNS) may result in patient 10 experiencing pain relief over a larger area, which may be advantageous in some instances. As previously discussed, fluid transfer device 106 may be implanted at various locations proximate to dorsal branches 22, 23 and perineal branches 24, 25. In any case, fluid transfer devices are typically positioned based on the perceived pain of patient 10 and the drugs delivered to treat the pain.

IMD 108 is also coupled to electrodes 104 via lead 102 in FIG. 8. In the example of FIG. 8, electrodes 104 are conventional ring electrodes. In other embodiments, the electrodes may be realized by one or more cuff electrodes, as shown in FIG. 11. In the illustrated example, electrodes 104 are connected to IMD 108 via internal electrical conductors within lead 102 and, optionally, a lead extension (not shown). The electrical stimulation delivered by electrodes 104 stimulates dorsal branch 22. In particular, electrodes 104 are shown implanted proximate to a portion of dorsal branch 22 prior to dorsal branch entering pudendal canal 14 in FIG. 8. Similar to fluid transfer device 106, positioning electrodes 104 higher along dorsal branch 22, may result in patient 10 experiencing paresthesia over a larger area.

System 100A generally operates in a similar manner to system 2 in FIG. 1 to apply electrical stimulation to patient 10 to treat a pelvic disorder such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region. However, unlike system 2, system 100A also delivers drug therapy in combination with electrical stimulation. Delivering drug therapy in combination with electrical stimulation may provide more complete pain relief for patient 10 or reduce and possibly prevent the affects of unwanted side effects.

External programmer 109 may be a small, battery-powered, portable device that may accompany patient 10 through the day. External programmer 109 may have a simple user interface, such as a button or keypad, and a display or lights. As shown, external programmer 109 may communicate via wireless communication with IMD 108. In particular, external programmer 109 may control delivery of drug therapy and electrical stimulation by IMD 108 using telemetry techniques known in the art. External programmer 109 may comprise a clinician programmer or a patient programmer. Where external programmer 109 comprises a patient programmer, patient 10 may only be able to activate and deactivate IMD 108. Where external programmer 109 comprises a clinician programmer, external programmer 109 may include additional functionality, e.g., menus for selecting parameter sets and programs and schedules for delivering the therapy according to the selected parameters sets and programs.

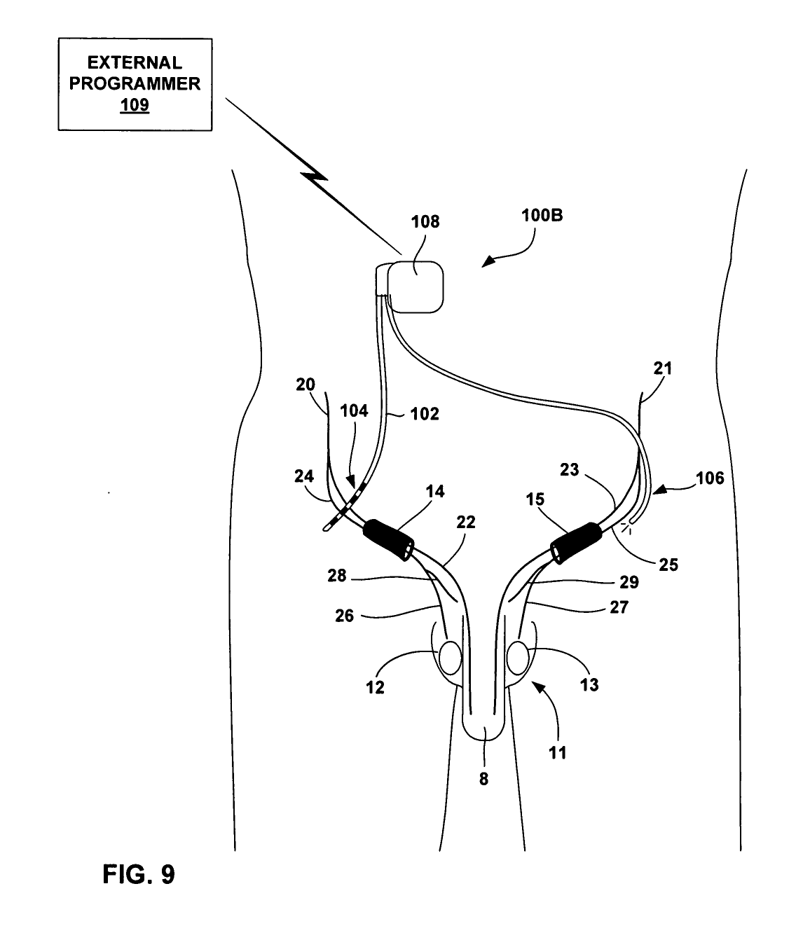

FIG. 9 is a schematic diagram illustrating another configuration for example system 100A of FIG. 8. In particular, in system 100B of FIG. 9, rather than being implanted along dorsal branch 22, electrodes 104 are illustrated in FIG. 9 as being implanted perpendicular to dorsal branch 22. Implanting electrodes 104 perpendicular to dorsal branch 22 may provide certain advantages. For example, when implanted as shown, electrodes 104 may more effectively apply electrical stimulation to a point along dorsal branch 22 instead of applying electrical stimulation along a length or portion of dorsal branch 22. Patient 10 may experience a more complete relief of pain or fewer unwanted side effects as a result of applying electrical stimulation in this manner. The invention is not limited to the illustrated embodiments. Instead, electrodes 104 may be implanted at any orientation with respect to dorsal branch 22.

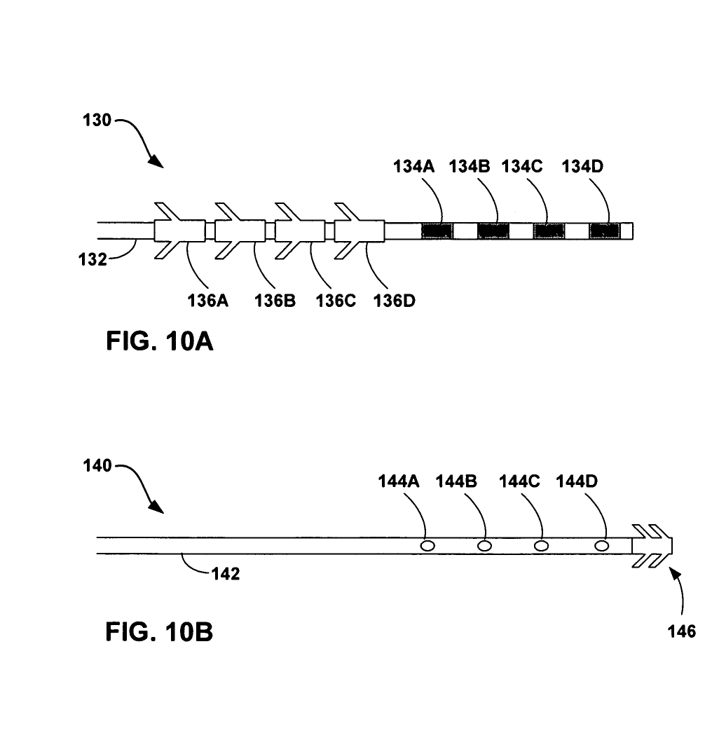

FIGS. 10A and 10B show exemplary electrical leads with fixation elements to secure the lead within a patient. As shown in FIG. 10A, lead 130 includes lead body 132, tines 136A-D (collectively tines 136) and electrodes 134A-D (collectively electrodes 134). Lead 130 may be a standard lead that includes all four tines 136 close to electrodes 134. Lead 130 may be implemented with any number of electrodes or tines. When implanting lead 130, having tines 136 close to electrodes 134 may be beneficial by allowing less movement of electrodes 134 with respect to a dorsal branch or perineal branch of a pudendal nerve.

Electrodes 134 are more effective in delivering electrical stimulation when the electrodes are located close to a dorsal branch or perineal branch. If electrodes 134 migrated away from a dorsal branch or perineal branch due to movement of the patient throughout the day, for example, the efficacy of the stimulation may decrease. Therefore, tines 136 located close to electrodes 134 may be beneficial to therapy efficacy. An arrangement of fixation elements similar to that shown in FIG. 10A may be provided on fluid transfer devices to anchor fluid outlets adjacent to target nerve sites.

FIG. 10B illustrates a fluid delivery device 140 which includes device body 142, tines 146, and lateral fluid outlets 144A-D (collectively outlets 144). Fluid delivery device 140 alternatively, or additionally, may include a distal outlet. Fluid delivery device 140 may be a standard fluid delivery device that includes tines 146 located at the distal end of device body 142. Fluid delivery device 140 may be implemented with any number of fluid outlets or tines. Fluid outlets 144 may be located close to or a distance away from tines 146. When fluid outlets 144 are close to tines 146, implanting fluid delivery device 140 may allow less movement of fluid outlets 144 with respect to a dorsal branch or perineal branch.

When fluid outlets 144 are located a distance away from tines 146, implanting fluid delivery device 140 may allow outlets 144 to reach further away from the anchoring site. For example, when fluid delivery device 140 delivers a drug to a dorsal branch or perineal branch, tines 146 may be anchored to tissue a distance away from the branch while outlets 144 may be located proximate to the branch. Securing tines 146 to a dorsal branch or perineal branch is undesirable because the nerve may be damaged in the process. Thus, fluid delivery device 140 may be beneficial by preventing unwanted nerve damage during the implantation process. An arrangement of fixation elements similar to that shown in FIG. 10B may be provided on electrical stimulation leads to anchor electrodes adjacent to target nerve sites.

FIG. 11 is a schematic diagram illustrating another exemplary arrangement for system 100A of FIG. 8 for delivering electrical stimulation in combination with drug therapy to male patient 10. In particular, system 100C is illustrated in FIG. 11 as including cuff electrode 105 deployed at the distal end of lead 102 instead of ring electrodes 104. In the illustrated example, cuff electrode 105 applies electrical stimulation to dorsal branch 22 of pudendal nerve 20 and fluid transfer device 106 delivers one or more drugs to perineal branch 25 of pudendal nerve 21 to treat a pelvic disorder of patient 10, such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region.

Cuff electrode 105 includes a cuff-like fixation structure and one or more electrodes carried by the fixation structure that deliver electrical stimulation to dorsal branch 22. Cuff electrode 105 may comprise a rigid cuff electrode, a self-sizing spiral cuff electrode, a half cuff electrode, a helical electrode, a chambered electrode, or other types of cuff electrodes that are shaped, sized and otherwise configured to at least partially wrap around dorsal branch 22. In general, cuff electrode 105 may be sized and shaped to at least partially enclose dorsal branch 22 and promote electrical coupling between the electrode and dorsal branch 22. Cuff electrode 105 may be sized and shaped to at least partially enclose dorsal branch 22 and promote electrical coupling pressure between the electrode and the nerve. Upon enclosure of at least a portion of a nerve branch, a cuff may be held in a closed position by shape memory properties, sutures, interlocking tabs, surgical adhesive, crimping, or other fixation techniques or structures. Cuff electrode 105 may include a single or multiple electrodes. For example, cuff electrode 105 may include a bipolar or multipolar arrangement of electrodes or a unipolar electrode that is referenced to the electrical potential of an active can electrode carried by IMD 108. For reference, FIGS. 6A-6C illustrate example cuff electrodes that may be useful in delivering electrical stimulation in combination with the described drug therapy and, more particularly, the fixation structure of such cuff electrodes.

A cuff electrode may provide more direct electrical contact with a branch of a pudendal nerve, i.e., a dorsal branch or a perineal branch, than a standard electrode lead. However, in some cases, applying electrical stimulation directly to a nerve may result in the patient experiencing an unpleasant sensation, such as a burning sensation. Consequently, a standard electrode implanted proximate to a branch of the pudendal nerve may be advantageous because the patient may experience a more pleasant paresthesia as a result of stimulation. In addition, a standard electrode lead may also be advantageous in terms of surgical ease.

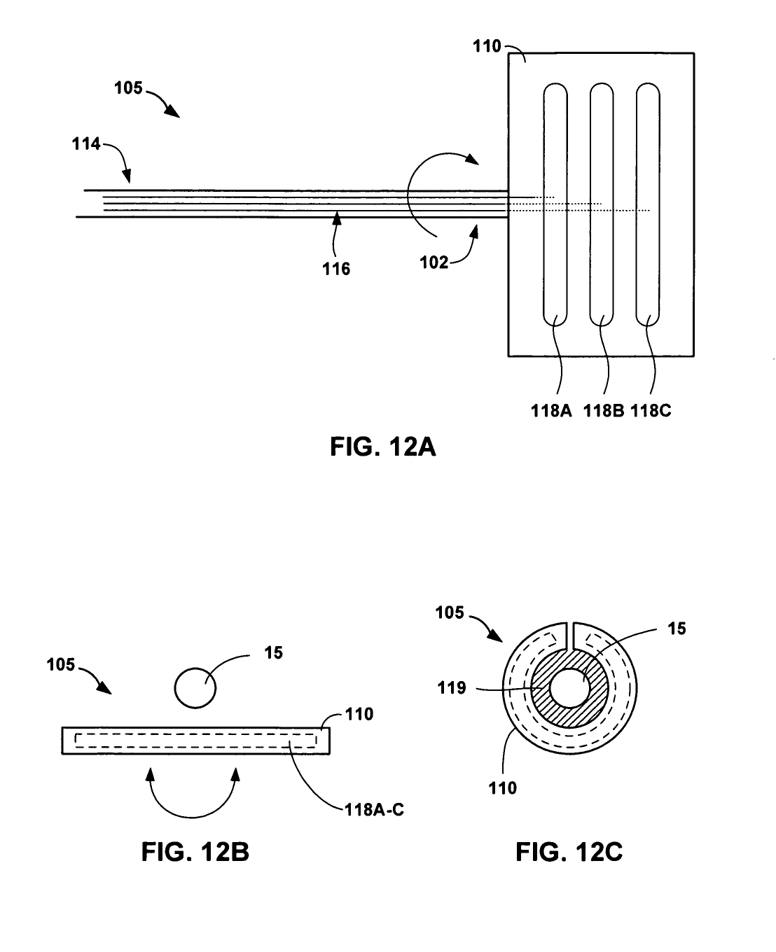

FIGS. 12A-12C are schematic diagrams illustrating an exemplary embodiment of cuff electrode 105. Cuff electrode 105 may be any type of cuff electrode used to deliver electrical stimulation, and may be deployed via lead 102 as shown in FIG. 11, either as an alternative to or in combination with other electrodes such as ring electrodes or paddle electrodes. In embodiments including more than one cuff electrode, the cuff electrodes may comprise the same type of cuff electrode or may comprise different types of cuff electrodes. In any case, cuff electrode 105 is merely exemplary and should not be considered limiting of the invention as broadly embodied and described in this disclosure. FIGS. 12A-12C illustrate the implantation of cuff electrodes to deliver electrical stimulation to branch of a pudendal nerve, i.e., a dorsal branch or a perineal branch.

FIG. 12A is a top view of cuff electrode 105. Cuff electrode 105 includes lead 102, fixation structure 110, a plurality of stimulation electrodes 118A-C, and a plurality of electrical conductors 116 within lead 18. In the example of FIG. 12A, cuff electrode 105 includes three electrodes 118A, 118B, 118C. In the illustrated example, electrodes 118A-C are arranged such that a major axis of each electrode extends laterally to a branch of a pudendal nerve, e.g., dorsal branch 22. In this manner, the length of each electrode may be wrapped about all or a portion of the circumference of the dorsal branch 22. The proximal end 114 of lead 102 is connected to IMD 108 and fixation structure 110 is attached to the distal end 112 of lead 18.

Cuff electrode 105 may generally include one electrode or a plurality of electrodes. Each of electrodes 118A-C is coupled to ground conductor 116 and at least one of supply conductors 116. Electrodes 118A-C may be driven together via a common conductor or independently via separate conductors. When electrodes 118A-C are driven by a common conductor, they may be referenced to one or more electrodes carried by another lead or one or more electrodes carried by the IMD housing. When electrodes 118A-C are driven by separate conductors, bipolar or multipolar electrode combinations may be formed on a single lead or among two or more leads, as well as between one or more leads and the IMD housing.

For a given bipolar pair of electrodes on a lead, one supply conductor sources stimulation energy to a first electrode and a second supply conductor sinks stimulation energy from a second electrode, with the stimulation energy propagating across nerve tissue between the first and second electrodes. Hence, one electrode may form a cathode while the other forms an anode. Also, in some embodiments, multiple anodes and cathodes may be used in an electrode combination. A switch device in the IMD determines which electrodes will function as cathodes and which electrodes will function as anodes.

Fixation structure 110 may be fabricated from a flexible biocompatible material that provides a flexible interface between the electrode and the dorsal branch 22. In some embodiments, fixation structure 110 may be fabricated from a rigid biocompatible material. The rigid fixation structure may form a split cylinder or a “U” shape sized to fit around the dorsal branch 22. In any case, when implanting electrode 110 the surgeon may elevate the dorsal branch 22 and wrap fixation structure 110 around the dorsal branch 22. The manner in which the surgeon installs cuff electrode 105 around dorsal branch 22 depends on the type of cuff electrode.

For example, if fixation structure 110 is fabricated from a shape memory alloy, fixation structure 110 may recover its shape at a fixed temperature, e.g., slightly under room temperature. By sufficiently cooling fixation structure 110, the surgeon can easily open the cuff and position fixation structure 110 under the target nerve branch. Because the nominal body temperature of the patient is above room temperature, fixation structure 110 warms up and recovers its initial shape thereby closing or wrapping fixation structure 110 around the nerve branch. In another example, the fixation structure may be constrained in flat manner using a surgical tool or hand and, when released, wraps around the nerve.

FIG. 12B is a cross sectional view of cuff electrode 105 implanted underneath dorsal branch 22. In the illustrated example, fixation structure 110 is generally flat thereby allowing the surgeon to easily position electrode 105 under dorsal branch 22. When fixation structure 110 is fabricated from a shape memory alloy material, the surgeon may cool fixation structure 110 prior to positioning fixation structure 110 to easily manipulate fixation structure 110 into the open configuration shown in FIG. 12B. The surgeon may then position fixation structure under dorsal branch 22. Fixation structure 110 will recover its initial shape, i.e., a substantially closed ring sized to fit around dorsal branch 22, as fixation structure warms up to its activation temperature.

FIG. 12C is a cross sectional via of cuff electrode 105 implanted and wrapped around dorsal branch 22. More specifically, FIG. 12C illustrates the shape of fixation structure 110 when it has returned to its initial shape in response to warming from the patient's body heat. In the illustrated example, a gap 119 exists between dorsal branch 22 and fixation structure 110. The gap may be filled with tissue or fluids and may provide a buffer that prevents cuff electrode 105 from damaging dorsal branch 22. Alternatively, fixation structure 110 may be sized to wrap around dorsal branch 22 such that there is no gap between fixation structure 110 and dorsal branch 22. In some embodiments, the fixation structure may be deployed using superelastic properties of a shape memory allow such as Nitinol. For example, the fixation structure may be constrained in a flat shape either manually or with a surgical tool, and then released so that it wraps around the nerve.

FIG. 13 is a schematic diagram further illustrating an example system 100D. In particular, system 100d is illustrated from the right side of a male patient 10 and includes leadless stimulators 150 and 151, e.g., as an alternative to a ring electrode lead or a cuff electrode lead. For purposes of illustration, FIG. 13 illustrates pudendal nerve 20, dorsal branch 22 and perineal branch 24 of pudendal nerve 20, pudendal canal 14, penis 8, scrotum 11, perineum 6, posterior scrotal branch 26 and muscular branch 28 of perineal nerve 24, and medial posterior scrotal branch 30 and lateral posterior scrotal branch 32 of posterior scrotal branch 26.

In the illustrated example, fluid transfer device 106 is implanted proximate to a portion of dorsal branch 22 at a point after dorsal branch 22 exits pudendal canal 14. Microstimulator 150 applies electrical stimulation to a portion of dorsal branch 22 at a point prior to dorsal branch 22 entering pudendal canal 14. Microstimulator 151 applies electrical stimulation to one or both of dorsal branch 22 and perineal branch via pudendal canal 14. In some cases, microstimulator 151 may apply electrical stimulation to both branches, i.e., dorsal branch 22 and perineal branch 24, because the branches are in close proximity to each other within pudendal canal 14. However, in some cases, microstimulator 151 may be oriented relative to pudendal canal, e.g., positioned at different points around the circumference of pudendal canal 14, such that electrical stimulation is applied substantially to only one of dorsal branch 22 and perineal branch 24.

In the following description, microstimulator will be described as indirectly delivering electrical stimulation to dorsal branch 22 via pudendal canal 14. In any case, fluid transfer device 106, microstimulator 150, and microstimulator 151 deliver drug therapy and electrical stimulation under control of IMD 108. In some embodiments, microstimulators 150 and 151 may be controlled by IMD 108 or external programmer 109 via wireless telemetry. In other embodiments, microstimulators 150 and 151 may operate autonomously, subject to reprogramming or parameter adjustment by external programmer 109.

As shown, IMD 108 or external programmer 109 may wirelessly control microstimulators 150 and 151 to deliver electrical stimulation to dorsal branch 22, directly and indirectly via pudendal canal 14, respectively. In the example of FIG. 13, microstimulators 150, 151 each include a housing 154, 157 and a fixation structure 152, 153, such as a cuff, attached to housing 154, 157, respectively. Housing 154, 157 may be formed into a capsule-like shape and may be constructed from any of a variety of biocompatible materials, such as titanium or stainless steel.

Housing 154, 157 may carry an implantable pulse generator (IPG) and a telemetry interface to exchange (send, receive, or both) control signals with IMD 108, external programmer 109, or both. Fixation structure 152, 153 wraps at least partially around dorsal branch 22 and pudendal canal 14, respectively, to secure microstimulator 150, 151 in place. Accordingly, fixation structure 152, 153 may operate and be constructed of a flexible or rigid biocompatible material similar to the fixation structure of previously described cuff electrode 104. Fixation structure 152, 153 may carry one or more electrodes, i.e., the electrodes may be integrated with fixation structure 152, 153, and housing 154, 157 may include short leads (not shown) that extend from housing 154, 157 to couple the electrodes to housing 154, 157, respectively. In some embodiments, housing 154, 157 may form an active “can” electrode.

Microstimulators 150, 151 may be implanted with less invasive procedures than electrodes that are coupled to an IMD via a lead. For example, because microstimulators 150, 151 wirelessly communicate with IMD 108, a surgeon does not have to tunnel a lead to IMD 108. In some embodiments, microstimulators 150, 151 may wirelessly communicate with external programmer 109.

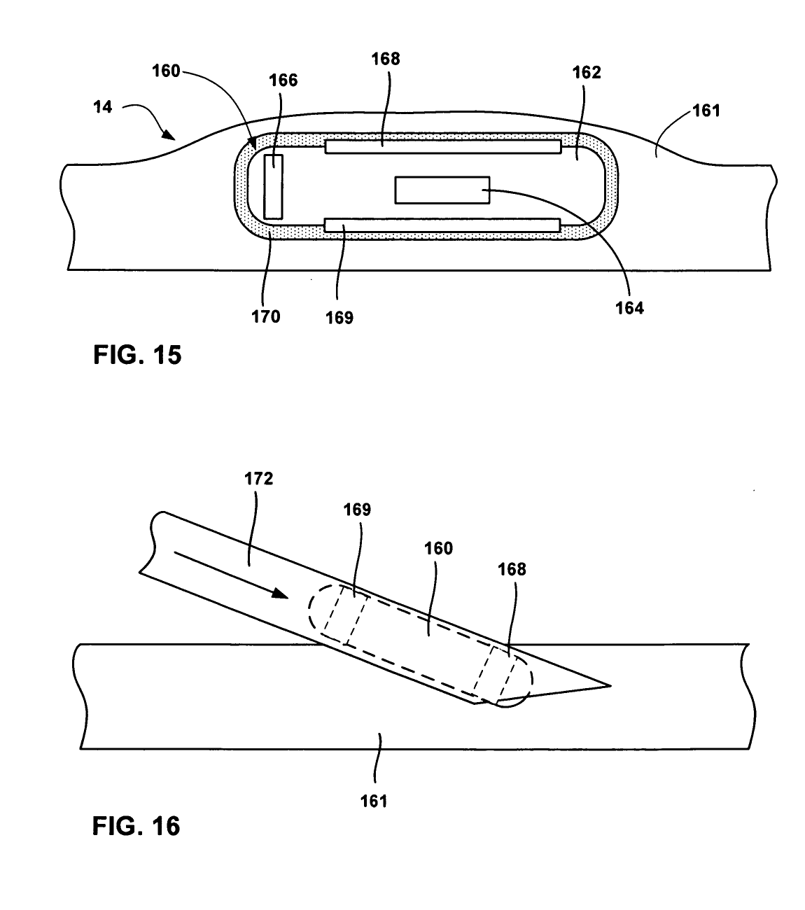

Microstimulators 150, 151 may also be implanted within tissue proximate to dorsal branch 22 or pudendal canal 14. In some cases, microstimulator 151 may be implanted within the external fascia of pudendal canal 14. In any case, microstimulators 150, 151 may be implanted in tissue using a needle (not shown) as illustrated in FIGS. 15 and 16. In this case, microstimulators 150, 151 may be implanted with a minimally invasive, percutaneous procedure. As an example, the needle may include a hollow cylinder and a pointed distal end for puncturing skin of patient 10. The needle may include the microstimulator and a fluid, e.g., saline solution, or push rod to force the microstimulator out of the needle. In this case, microstimulators 150, 151 may be miniaturized in order to be implanted using the needle. In some embodiments, a plurality of microstimulators may be implanted within tissue proximate to dorsal branch 22 or within pudendal canal 14. The plurality of implanted microstimulators may apply electrical stimulation independently or on a coordinated basis.

When implanted within tissue, microstimulators 150, 151 may comprise a self-contained module. The module comprises a housing that may carry one or more electrodes and an IPG within the housing. The IPG may comprise a circuit board and a power source, such as a battery, to provide power to the circuit board and electrodes. The circuit board may include the telemetry interface and other processing electronics. The electrodes may be pads mounted on a surface of the housing or ring electrodes that extend about the entire periphery of the housing. In some cases, the housing itself may form an active “can” electrode in addition to the electrodes mounted on the housing.

The invention is not limited to the illustrated configuration. In general, fluid transfer device 106 and microstimulators 150, 151 may be implanted in any combination to deliver drug therapy in combination with electrical stimulation to at least one of dorsal branches 22, 23, and perineal branches 24, 25. Furthermore, any number of fluid transfer devices and microstimulators or other types of electrodes may be implanted in any combination to provide uni-lateral or bi-lateral pain relief.

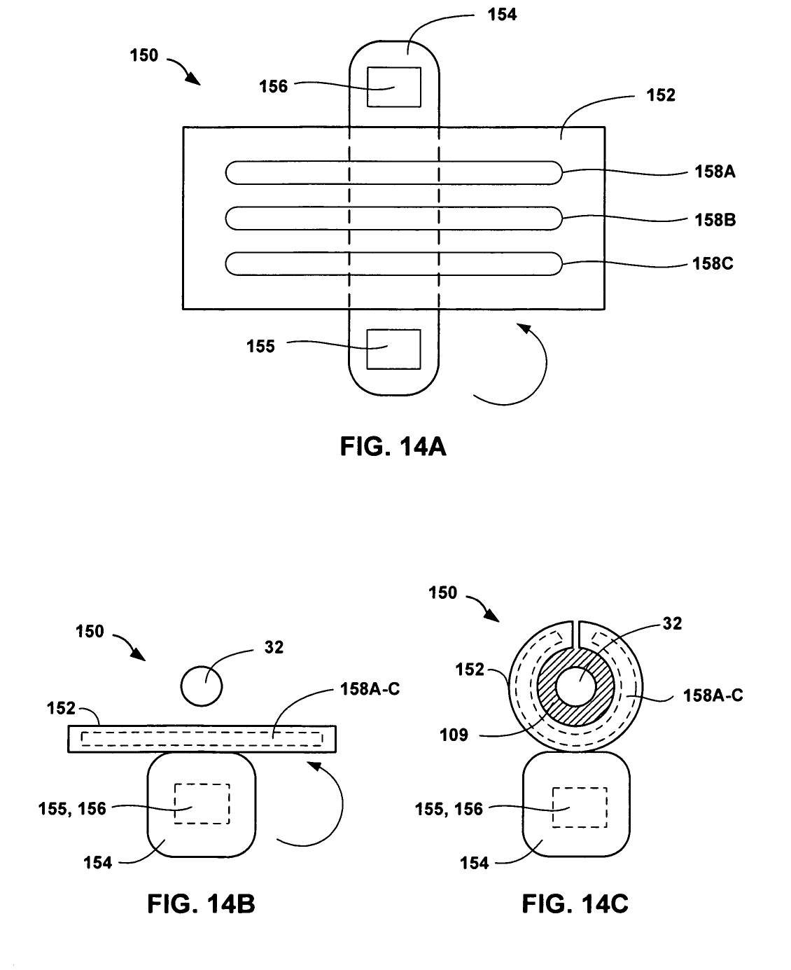

FIGS. 14A-14C are enlarged schematic diagrams showing microstimulator 150. Although FIGS. 14A-14C illustrate microstimulator 150, microstimulator 156 may be constructed and operate in the same manner. In particular, FIG. 14A is an enlarged top view of microstimulator 150 including housing 154, circuit board 156, power supply 155, fixation structure 152, and electrodes 158A-C (collectively electrodes 158). Housing 154 may have a rounded, capsule-like shape, and a smooth, atraumatic surface formed of one or more biocompatible materials, such as titanium, stainless steel, epoxy, or polyvinylchloride. However, the invention is not so limited. Instead, housing 154 may have a shape that is compatible with the anatomy at the implant site, i.e., at various locations along a dorsal or perineal branch of a pudendal nerve of a patient. In some embodiments, the leadless microstimulator may have a capsule shape with a diameter of approximately less than or equal to approximately 2 cm and a length of less than or equal to approximately 5 cm.

Fixation structure 152 may be constructed of a flexible or rigid biocompatible material that at least partially wraps around, for example, the dorsal nerve branch, e.g., like a cuff. For example, fixation structure 152 may be fabricated from a shape memory alloy that has the capacity to recover a memorized shape when deformed at a certain temperature and then heated at a higher temperature or vice versa. In this case, the memorized shape may be a split cylinder or a substantially closed cylinder with a diameter sized to wrap around the dorsal nerve branch.

FIG. 10A illustrates fixation structure 152 in a deformed, generally open state that enables a surgeon to easily position slip microstimulator 150 underneath dorsal nerve branch 22. However, after positioning microstimulator 150 beneath dorsal nerve branch 22, the body temperature of the patient causes fixation structure 152 to recover its memorized shape, i.e., a split cylinder. Therefore, fixation structure 152 may be beneficial by reducing trauma during surgical implantation procedures.

Fixation structure 152 also carries one or more electrodes 158. Electrodes 158 may be driven together or independently. Electrodes 158 may be integrated with fixation structure 152 or, alternatively housing 154 may include short leads (not shown) that extend from housing 154 to couple electrodes 158 to housing 154.

Circuit board 156 may include a processor, memory, pulse generator circuitry to generate electrical pulses delivered by IMD 108, and telemetry circuitry for wireless telemetry with IMD 108, external programmer 109, or both. As an example, the memory may store stimulation parameters, e.g., electrode polarity, pulse width, pulse rate, and amplitude. Memory may also store schedules which define times for the processor to select particular parameters. A schedule may cause electrical stimulation to be delivered at respective times. In this manner, the processor may control the pulse generator circuitry generate electrical stimulation pulses in accordance with the selected parameters and schedule.

Microstimulator 150 may also operate under control from an external programmer, so that a physician or patient may activate, deactivate and/or modify stimulation delivered to the patient on a selective basis. Power source 155 supplies operating power to circuit board 156 and may take the form of a small rechargeable or non-rechargeable battery. Different types of batteries or different battery sizes may be used. To promote longevity, power source 155 may be rechargeable via induction or other means.

FIG. 14B illustrates a cross sectional view of microstimulator 150 implanted underneath dorsal nerve branch 22. In the illustrated example, fixation structure 152 is flat, thereby allowing the surgeon to easily position microstimulator 150 underneath dorsal nerve branch 22. When fabricated from a shape memory alloy, the body temperature of patient 10 may heat fixation structure 152 above the recovery shape temperature.

FIG. 14C is a cross sectional view of microstimulator 150 with fixation structure 152 wrapped substantially around dorsal nerve branch 22. For example, as fixation structure 152 is warmed above its recovery shape temperature, fixation structure 152 recovers its initial shape, i.e., a substantially closed cylinder or ring. As shown in FIG. 14C, in some embodiments, fixation structure 152 may not close completely. However, fixation structure 152 may at least wrap partially around dorsal nerve branch 22 in order to secure microstimulator 150 to the nerve site. Removing microstimulator 150 may be easier when fixation structure 152 does not completely wrap around dorsal nerve branch 22 because the gap between the ends of fixation structure 152 may provide an area to insert a tool that aids in removal. In alternative embodiments, fixation structure 152 may wrap completely around dorsal nerve branch 22.

In the illustrated example, a gap 109 exists between dorsal nerve branch 22 and fixation structure 152. Gap 109 may be filled with tissue or fluids and may provide a buffer that prevents microstimulator 150 from damaging dorsal nerve branch 22. Alternatively, fixation structure 152 may be sized to wrap around dorsal nerve branch 22 such that there is no gap between fixation structure 152 and dorsal nerve branch 22.

FIG. 15 is cross-sectional view of a microstimulator 160 implanted within, for example, tissue 161 of a pudendal canal, e.g., pudendal canal 14, of a patient. Microstimulator 160 may also be implanted in tissue proximate to a dorsal or perineal branch of a pudendal nerve of a patient. Housing 162 of microstimulator 160 is embedded in tissue 161 of pudendal canal 14 and includes circuit board 164, power source 166, and electrodes 168 and 169. Housing 162 is in the shape of a rounded capsule and includes a smooth surface. The only structure extending from housing 162 are electrodes 168 and 169. Electrodes 168 and 169 may protrude slightly from housing 162 or, alternatively, may be integrated into housing 162 to apply electrical stimulation to tissue 161. Microstimulator 160 rests in wall cavity 170 formed within tissue 161. As previously described, microstimulator 160 may have a cylindrical shape with a diameter of less than or equal to approximately 2 cm and a length of less than or equal to approximately 5 cm.

Circuit board 164, power source 166, and electrodes 168 and 169 may be similar to respective circuit board 156, power source 155, and electrodes 158 of FIGS. 14A-14C. Differences between these components may relate to the size or shape of each component. Therefore, electrodes 168 and 169 apply electrical stimulation under control of circuit board 164. Power source 155 supplies operating power to circuit board 164. Circuit board 164 may select may select stimulation parameters and cause electrodes 168 and 169 to apply electrical pulses with the selected parameters according to schedules stored in memory. Circuit board 160 receives control signals from IMD 108, external programmer 109, or both by wireless telemetry. In some embodiments, one of electrodes 168 and 169 may comprise a sensor or microstimulator 160 may additionally include a sensor that detects a physiological parameter. In such embodiments, the sensor may sense a change in a physiological parameter. Processing electronics on circuit board 164 detects the change and causes electrodes 168 and 169 to apply electrical stimulation in response to the change.

Implanting microstimulator 160 within tissue 161 of pudendal canal 14 may be a simple method for securing electrodes 168 and 169. In some embodiments, a plurality of microstimulators similar to microstimulator 160 may be implanted and indirectly apply electrical stimulation to a dorsal nerve branch, a perineal nerve branch, or both via pudendal canal 14 in a coordinated manner or in a manner independent of each other.

FIG. 16 is a schematic diagram illustrating implantation of microstimulator 160 within tissue 161 of pudendal canal 14. Microstimulator 160 may be implanted through endoscopic, laparoscopic, or similar minimally invasive techniques. A surgeon may make a small incision as in a pudendal neurectomy procedure in patient 10 and guides microstimulator 160 within needle 172 to tissue 161. Needle 172 may be constructed of a metal alloy and comprise a hollow cylinder and a pointed distal end for puncturing the skin of patient 10. Needle 172 includes microstimulator 160 and a fluid or push rod to force microstimulator 160 out of the needle. An exemplary fluid may be saline or other biocompatible fluid.

Once needle 172 in positioned at the appropriate location with respect to the target nerve branch, the surgeon may force microstimulator 160 into place. Removing needle 172 from tissue 161 allows tissue 161 to close and surround microstimulator 160. When implanting microstimulator 160, the tissue 161 should not be breached in order to prevent pudendal canal 14 from being damaged.

In other embodiments, microstimulator 160 may be implanted through more invasive procedures. As previously described, multiple microstimulators may be implanted in a pudendal canal or tissue proximate to a pudendal canal, dorsal nerve branch, or perineal nerve branch to apply electrical stimulation to a larger area.

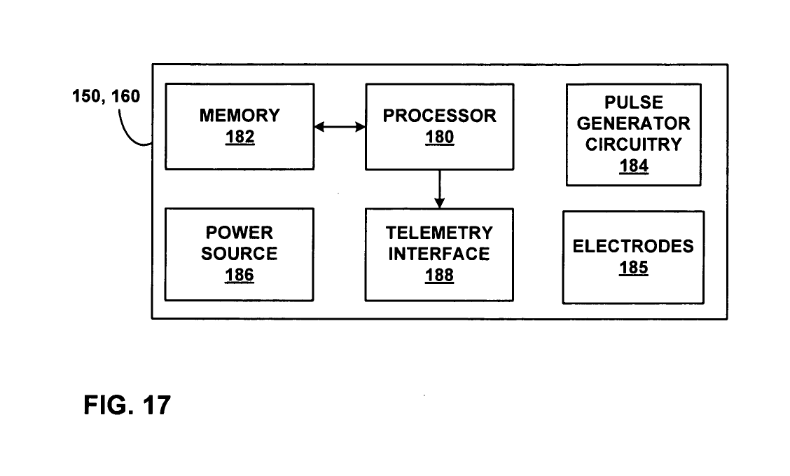

FIG. 17 is a functional block diagram illustrating various components of an example microstimulator 150, 151 (FIG. 13) or microstimulator 160 (FIGS. 15 and 16). In the example of FIG. 17, microstimulators 150, 151 and 160 include a processor 180, memory 182, pulse generator circuitry 184, telemetry interface 188, power source 186 and electrodes 185. Pulse generator circuitry 184 may be carried on a circuit board, along with processor 180, memory 182, and telemetry interface 188. Memory 182 may store instructions for execution by processor 180, stimulation parameters, e.g., electrode polarity, pulse width, pulse rate, and amplitude, and schedules for delivering electrical stimulation. Memory 182 may include separate memories for storing instructions, stimulation parameter sets, and schedules. Memory 182 may comprise any form of computer-readable media such as magnetic or optical tape or disks, solid state volatile or non-volatile memory, including random access memory (RAM), read only memory (ROM), electronically programmable memory (EPROM or EEPROM), or flash memory.

Processor 180 controls pulse generator circuitry 184 to deliver electrical stimulation via electrodes 185. Electrodes 185 may comprise any number and type of electrodes previously described, i.e., electrodes 158 (FIG. 13) and electrodes 168 and 169 (FIGS. 15 and 16). Electrical stimulation may be applied with various ranges of stimulation pulse parameters for treating pelvic disorders, such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region. Using chronic pelvic pain, including urogenital pain such as chronic groin pain, chronic testicular pain (CTP), prostatitis-like pain, or pain associated with PNE as an example, an exemplary range of stimulation pulse parameters likely to be effective are as follows: pulse widths between approximately 10 and 5000 microseconds, more preferably between approximately 100 and 1000 microseconds and still more preferably between 180 and 450 microseconds; voltage amplitudes between approximately 0.1 and 50 volts, more preferably between approximately 0.5 and 20 volts and still more preferably between approximately 1 and 10 volts; and frequencies between approximately 0.5 and 500 hertz, more preferably between approximately 10 and 250 hertz and still more preferably between approximately 50 and 150 hertz. The pulses may be alternating current (ac) pulses or direct current (dc) pulses, and may be mono-phasic, bi-phasic, or multi-phasic in various embodiments. The above parameters may be applicable to stimulation delivered by microstimulators, paddle lead electrode arrays, ring electrode leads, or other stimulation electrodes.

Processor 180 also controls telemetry interface 188 to receive information from IMD 108, external programmer 109, or both. Telemetry interface 188 may communicate via wireless telemetry, e.g., RF communication, on a continuous basis, at periodic intervals, or upon request from the implantable stimulator or programmer. Processor 180 may include a single or multiple processors that are realized by microprocessors, Application-Specific Integrated Circuits (ASIC), Field-Programmable Gate Arrays (FPGA), or other equivalent integrated or discrete logic circuitry.

Power source 186 delivers operating power to the components of the implantable microstimulator. As mentioned previously, power source 186 may include a small rechargeable or non-rechargeable battery and a power generation circuit to produce the operating power.

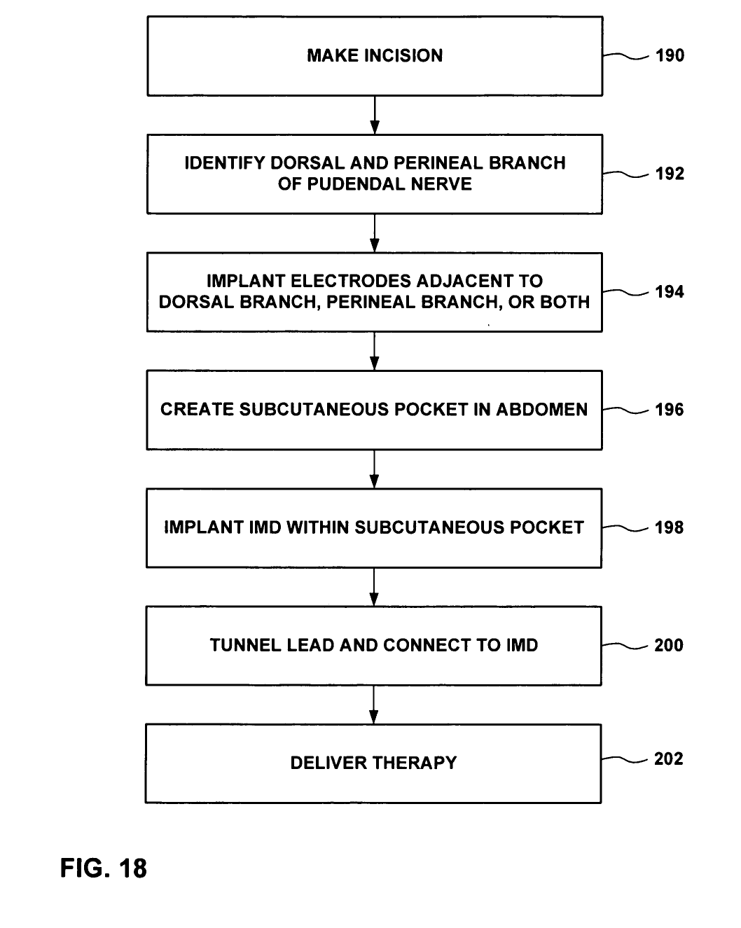

FIG. 18 is a flow chart illustrating a technique for applying electrical stimulation to at least one branch of a pudendal nerve of a patient, e.g., a dorsal branch, a perineal branch, or both, using an IMD including an electrical stimulation device. The IMD may include any of the previously described electrodes, i.e., electrodes carried by a lead (FIGS. 1-5, 8 and 9), cuff electrodes (FIG. 11), and microstimulators (FIGS. 13, 15, and 16). In some embodiments, the IMD may also include a drug delivery device. In such embodiments, the IMD may include any number of fluid transfer devices implanted to deliver drug therapy in combination with electrical stimulation in accordance with the steps of the illustrated flowchart. The flow of events begins with the surgical procedure for implanting the electrodes. A surgical procedure such as those used in exposing the pudendal nerve, implanting stimulation electrodes for treating sexual dysfunction, pudendal denervation, or other procedures that expose the dorsal and perineal branches of a pudendal nerve of a patient may be used. Specifically, the surgeon may make an incision (190) similar to that used for standard pudendal denervation.

The surgeon identifies the dorsal branch and perineal branch of a pudendal nerve (192) and implants an electrode adjacent to the dorsal branch, perineal branch, or both (194). In some embodiments, the surgeon may implant the electrodes within the pudendal canal or tissue proximate to the pudendal canal to deliver electrical stimulation indirectly to one or more of the dorsal and perineal branch of a pudendal nerve. Where the lead carrying the electrodes includes fixation elements, such as tines, barbs, and other anchoring devices, the surgeon may secure the fixation elements to tissue adjacent to the nerves to avoid damage to the nerve and prevent the fluid transfer device from shifting as the patient moves. If the lead includes a fixation element similar to the cuff of cuff electrode 105 (FIGS. 12A-12C), the surgeon may elevate the nerve and wrap the cuff around the nerve. If the fixation structure is formed from a shape memory alloy, the body temperature of the patient may cause the fixation structure to recover its initial shape, i.e., a substantially closed cylinder or ring shape sized to fit around the nerve. In any case, the cuff may wrap at least partially around the nerve thereby securing the fluid transfer device to the nerve.

Leads carrying electrodes may provide distinct advantages due to the number of electrodes available to apply electrical stimulation. For example, leads are available that carry eight, sixteen, or more electrodes which can be used to apply electrical stimulation in various groups or independently of each other. Further, because the electrodes may be positioned along a substantial length of the lead, the electrodes may apply electrical stimulation along a larger area of the nerve.

In some embodiments, the surgeon may implant microstimulator 150 (FIG. 13) similar to cuff electrode 105 (FIG. 11) because the fixation structure of microstimulator 150 may operate in the same manner as the fixation structure of cuff electrode 105. In contrast, the surgeon may implant microstimulator 160 (FIGS. 15 and 16) within the external fascia of the pudendal canal using a needle. The needle may comprise a hollow cylinder and a pointed distal end for puncturing the skin of the patient and a fluid to force microstimulator 140 out of the needle. Accordingly, the surgeon may not need to make an incision when implanting microstimulator 140 within the external fascia of the pudendal canal. Rather, once the needle is positioned at the appropriate location with respect to the pudendal canal, the surgeon forces microstimulator 140 into place by depressing the plunger of the needle thereby forcing the fluid and microstimulator out of the needle.

Removing the needle from the pudendal canal allows the external fascia of the spermatic cord to close and surround microstimulator 140. Consequently, microstimulator 140 may be implanted with a minimally invasive surgical procedure. Additionally, in some embodiments, the surgeon may implant a plurality of microstimulators along the pudendal canal or within tissue along a dorsal branch or perineal branch of a pudendal nerve. The microstimulators may provide electrical stimulation independently or on a coordinated basis.

The implantation techniques may be used for implanting electrodes at various locations along a dorsal branch or perineal branch of a pudendal nerve, e.g., at a point prior to the branch entering the pudendal canal or after the branch exits the canal. Electrodes may also, in some embodiments, be implanted proximate to one or both of dorsal and perineal branches within the pudendal canal.

In embodiments in which drug therapy is delivered to a branch of a pudendal nerve, i.e., a dorsal or perineal branch, in combination with electrical stimulation, the surgeon may implant fluid transfer devices using a method similar to implanting electrodes. For example, when implanting a fluid transfer device, fixation elements may secure the fluid transfer device to tissue proximate to the nerve branch. Leads carrying electrodes may provide distinct advantages over leadless stimulators due to the number of electrodes available to apply electrical stimulation. For example, leads are available that carry eight, sixteen, or more electrodes which can be used to applying electrical stimulation in various groups or independently of each other. Further, because the electrodes may be positioned along a substantial length of the lead, the electrodes may apply electrical stimulation along a larger area of the target nerve branch.

In any case, after implanting the electrodes, the surgeon may create a subcutaneous pocket in the abdomen of the patient (196) and implant an IMD, such as IMD 4 (FIGS. 1-5) or IMD 108 (FIGS. 8, 9, 11, and 13), within the subcutaneous pocket (198). In some embodiments, the IMD may be miniaturized and implanted within the scrotum of the patient. The surgeon may then tunnel the electrode lead through the patient to the implantation site and connect the lead to the IMD (200). Notably, in embodiments that deliver electrical stimulation in combination with drug therapy, microstimulators 150, 151 and 160 may wirelessly communicate with external programmer 109 to receive control signals and, thus, do not require an IMD.

When the surgical implantation procedure is complete, the implanted electrodes may deliver electrical stimulation (202) to at least one of a dorsal or a perineal branch of a pudendal nerve. Applying electrical stimulation to the branches of a pudendal nerve may treat a pelvic disorder such as sexual dysfunction, urinary incontinence, pudendal nerve entrapment (PNE), chronic groin pain, chronic testicular pain (CTP), prostatitis-like pain, and urogenital pain or other forms of pelvic pain that cause long term (chronic) pain in the pelvic or groin region. Using chronic pain as an example, the pain experienced by the patient may be uni-lateral or bi-lateral. Consequently, electrodes may be implanted adjacent to at least one branch of one or both pudendal nerves of a patient. The pain experienced by the patient may also be constant or intermittent, or spontaneous or exacerbated by physical activities and pressure. Thus, the implanted electrodes may apply electrical stimulation on demand, such as in response to a control signal received from a patient or clinician programmer, or in accordance with preprogrammed cycles or schedules.

Electrical stimulation of the dorsal or perineal branch of a pudendal nerve may treat sexual dysfunction and/or urinary incontinence in men and women by providing additional stimulation to nerves. For example, the applied stimulation may aid or enhance the ability of a male to create and sustain an erection or, in women, aid or enhance the ability to produce lubrication and orgasm or alleviate pain associated with sex pain disorders (dyspareunia and vaginismus). In another example, the applied stimulation may aid or enhance the ability of a male or female to control nerves to store and release urine. Electrical stimulation may also provide substantial relief of pelvic pain experienced by male and female patients, including urogenital pain, chronic groin pain, chronic testicular pain (CTP), prostatitis-like pain, and pain associated with PNE or other forms of pelvic pain that cause chronic pain in the pelvic or groin region may be caused by a variety of injuries or disorders in men and women.

The invention is not limited to delivering only electrical stimulation. Rather, the invention also describes embodiments that deliver drug therapy in combination with electrical stimulation to at least one of a dorsal branch and a perineal branch of one or both pudendal nerves. Electrical stimulation and drug therapy may be delivered simultaneously or on an alternating basis. For example, drug therapy may be delivered constantly or intermittently through the course of a day and the patient may use a patient programmer to deliver electrical stimulation when experiencing moments of increased pain. Alternatively, electrical stimulation may be delivered according to preprogrammed parameter sets and schedules and the patient may use a patient programmer to deliver drug therapy when the electrical stimulation does not substantially reduce the pain.

The techniques described in this disclosure may be implemented in hardware, software, firmware or any combination thereof. For example, various aspects of the techniques may be implemented within one or more microprocessors, digital signal processors (DSPs), application specific integrated circuits (ASICs), field programmable logic arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry.

When implemented in software, the functionality ascribed to the systems and devices described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic media, optical media, or the like. The instructions are executed to support one or more aspects of the functionality described in this disclosure

Many embodiments of the invention have been described. Various modifications may be made without departing from the scope of the claims. For example, although delivery of one or more drugs has been described, other fluids may be delivered in addition, or as an alternative, to such drugs. Such fluids may include, for example, saline, biological fluids, gene therapy suspensions or cultures, or the like. These and other embodiments are within the scope of the following claims.

1. A method comprising applying electrical stimulation to at least one branch of a pudendal nerve of a patient via an implanted electrical stimulation device.

2. The method of claim 1, wherein the pudendal nerve includes a dorsal branch and a perineal branch.

3. The method of claim 2, further comprising applying the electrical stimulation to at least one of the dorsal and perineal branches of the pudendal nerve at a point prior to the nerve branch entering a pudendal canal of the patient.

4. The method of claim 2, further comprising applying the electrical stimulation to one of the dorsal and perineal branches of the pudendal nerve at a point after the nerve branch exits a pudendal canal of the patient.

5. The method of claim 1, further comprising applying electrical stimulation to at least one branch of the pudendal nerve via a pudendal canal of the patient.

6. The method of claim 1, further comprising applying electrical stimulation to at least one of a dorsal branch and a perineal branch of first and second pudendal nerves of the patient via the implanted electrical stimulation device.

7. The method of claim 6, further comprising applying the electrical stimulation to at least one of the dorsal and perineal branches of the first and second pudendal nerves at points prior to the nerve branches entering respective pudendal canals of the patient.

8. The method of claim 6, further comprising applying the electrical stimulation to at least one of the dorsal and perineal branches of the first and second pudendal nerves at points after the nerve branches exit respective pudendal canals of the patient.

9. The method of claim 6, further comprising applying the electrical stimulation to at least one of the dorsal and perineal branches of the first and second pudendal nerves via first and second pudendal canals of the patient.

10. The method of claim 1, wherein the electrical stimulation device delivers electrical stimulation selected to treat one or more pelvic disorders.

11. The method of claim 10, wherein the pelvic disorders include at least one of pudendal nerve entrapment (PNE), chronic groin pain, chronic testicular pain (CTP), prostatitis-like pain, urogenital pain, sexual dysfunction, and urinary incontinence.

12. The method of claim 1, wherein the electrical stimulation device comprises one or more of a cuff electrode, a ring electrode, a planar electrode, or an electrode on a leadless stimulator.

13. The method of claim 1, further comprising delivering a drug to at least one of a dorsal branch and a perineal branch of the pudendal nerve of the patient via an implanted drug delivery device.

14. The method of claim 13, wherein delivering the drug comprises delivering the drug to at least one of a dorsal branch and a perineal branch of first and second pudendal nerves of the patient via the implanted drug delivery device.

15. The method of claim 13, wherein the drug is selected to treat a pelvic disorder.

16. A system comprising:
an implantable electrical stimulation device that generates electrical stimulation selected to treat a pelvic disorder; and
one or more electrodes coupled to the electrical stimulation device at a position adjacent to at least one of a dorsal branch and a perineal branch of a pudendal nerve of a patient.

17. The system of claim 16, wherein the electrical stimulation is selected to treat the pelvic disorder including at least one of pudendal nerve entrapment (PNE), chronic groin pain, urogenital pain, chronic testicular pain (CTP), prostatitis-like pain, sexual dysfunction, and urinary incontinence.

18. The system of claim 16, wherein the one or more electrodes are positioned to apply the electrical stimulation to at least one of the dorsal and perineal branches of the pudendal nerve at a point prior to the branches entering a pudendal canal of the patient.

19. The system of claim 16, wherein the one or more electrodes are positioned to apply the electrical stimulation to at least one of the dorsal and perineal branches of the pudendal nerve at a point after the branches exit a pudendal canal of the patient.

20. The system of claim 16, wherein the one or more electrodes are positioned to apply the electrical stimulation to at least one of the dorsal and perineal branches of the pudendal nerve via a pudendal canal of the patient.

21. The system of claim 16, wherein the one or more electrodes are positioned to apply the electrical stimulation to at least one a dorsal branch and a perineal branch of first and second pudendal nerves of the patient.

22. The system of claim 16, wherein the electrodes include at least one of a cuff electrode, a ring electrode, a planar electrode or an electrode on a leadless stimulator.

23. The system of claim 16, further comprising an implantable drug delivery device that delivers a drug to at least one of the dorsal branch and the perineal branch of the pudendal nerve of the patient.

24. The system of claim 23, wherein the implantable drug delivery device delivers the drug to at least one of a dorsal branch and a perineal branch of first and second pudendal nerves of the patient.

25. A method comprising:
delivering electrical stimulation to at least one of a dorsal branch and a perineal branch of at least one pudendal nerve of a patient via an implanted electrical stimulation device; and
delivering a fluid to at least at least one of the dorsal and perineal branches of the pudendal nerves of the patient via an implanted fluid delivery device,
wherein the implanted fluid delivery device and the implanted fluid delivery device share a common housing.

26. The method of claim 25, wherein delivering a fluid includes delivering a drug via a catheter coupled to the common housing, and delivering electrical stimulation includes delivering the electrical stimulation via a lead coupled to the common housing.

27. The method of claim 25, wherein the electrical stimulation and fluid are selected to treat a pelvic disorder including at least one of pudendal nerve entrapment (PNE), chronic groin pain, urogenital pain, chronic testicular pain (CTP), prostatitis-like pain, sexual dysfunction, and urinary incontinence.

28. A system comprising:
an implantable electrical stimulation device that delivers electrical stimulation selected to treat a pelvic disorder to at least one of a dorsal branch and a perineal branch of at least one pudendal nerve of a patient; and
an implantable fluid delivery device that delivers a fluid selected to alleviate a pelvic disorder to at least one of the dorsal and perineal branches of at least one pudendal nerve of the patient,
wherein the implanted electrical stimulation device and the implanted fluid delivery device share a common housing.

29. The system of claim 28, further comprising a lead coupled to the common housing to deliver the electrical stimulation, and a catheter coupled to the common housing to deliver the fluid.

30. A method comprising delivering a fluid to at least at least one of the dorsal and perineal branches of the pudendal nerves of the patient via an implanted fluid delivery device.

31. The method of claim 30, wherein delivering a fluid includes delivering a drug via a catheter coupled to implanted fluid delivery device.

32. The method of claim 30, wherein the fluid is selected to treat a pelvic disorder including at least one of pudendal nerve entrapment (PNE), chronic groin pain, urogenital pain, chronic testicular pain (CTP), prostatitis-like pain, sexual dysfunction, and urinary incontinence.

33. A system comprising:
an implantable fluid delivery device that containing a fluid selected to alleviate a pelvic disorder; and
a catheter, coupled to the implantable fluid delivery device, that delivers the fluid to at least one of the dorsal and perineal branches of at least one pudendal nerve of the patient.

34. The system of claim 33, wherein the fluid is a drug selected to treat a pelvic disorder including at least one of pudendal nerve entrapment (PNE), chronic groin pain, urogenital pain, chronic testicular pain (CTP), prostatitis-like pain, sexual dysfunction, and urinary incontinence.
